(19) United States
(12) Patent Application Publication
(10) Pub. No.: US 2009/0042878 A1
(43) Pub. Date: Feb. 12, 2009

(54) THIENOPYRIMIDYLAMINES AS MODULATORS OF THE EP2 RECEPTOR

(76) Inventors: Nico BRAEUER, Jena (DE); Bernd Buchmann, Hohen Neuendorf (DE); Marcus Koppitz, Berlin (DE); Olaf Peters, Tabarz (DE); Antonius Ter Laak, Berlin (DE); Bernhard Lindenthal, Berlin (DE); Gernot Langer, Falkensee (DE); Tim Wintermantel, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/170,774

(22) Filed: Jul. 10, 2008

Related U.S. Application Data
(60) Provisional application No. 60/949,341, filed on Jul. 12, 2007.

(30) Foreign Application Priority Data
Jul. 12, 2007 (EP) 07 075 603.6

(51) Int. Cl. A61K 31/5377; C07D 495/04; A61K 31/519; A61P 35/00

(52) U.S. Cl. 514/234.2; 544/278; 514/260.1; 544/117

(57) ABSTRACT

The present invention relates to thienopyrimidylamines of the general formula I, to processes for their preparation and to their use for production of pharmaceutical compositions for treatment of disorders and indications connected to the EP₂ receptor.

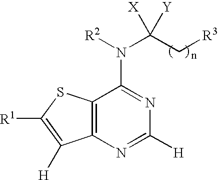

This application claims the benefit of the filing date of U.S. Provisional Application Ser. No. 60/949,341 filed Jul. 12, 2007, which is incorporated by reference herein.

The present invention relates to thienopyrimidylamines as EP₂ receptor modulators, to processes for their preparation and to their use as medicaments.

It has long been known that prostaglandins are key molecules in the processes of female reproductive biology such as, for example, control of ovulation, of fertilization, of nidation, of decidualization (e.g. placenta formation) and of menstruation. Prostaglandins likewise play an important part in the pathological changes in the reproductive tract, including menorrhagia, dysmenorrhea, endometriosis and cancer. The mechanism by which prostaglandins bring about these changes has not yet been completely elucidated. Recent results indicate that prostaglandins, their receptors and signal transduction pathways thereof are involved in processes such as angiogenesis, apoptosis, proliferation, and in inflammatory/antiinflammatory and immunological processes.

The effects of prostaglandins are mediated by their G protein-coupled receptors which are located on the cell surface. Prostaglandin E₂ (PGE₂) is of particular interest, having a wide variety of cellular effects through binding to functionally different receptor subtypes, namely the EP₁, EP₂, EP₃ and EP₄ receptors. The receptor subtypes to which prostaglandin E₂ binds appear to be of particular interest for the receptor-mediated effects which are involved in the control of fertility. It has thus been possible to show that the reproductive functions in EP₂ knockout mice (EP₂^{-/-}), i.e. in mice no longer having a functional PGE₂ receptor of the EP₂ subtype, are impaired, and that these animals have a smaller “litter size” (Matsumoto et al., 2001, Biology of Reproduction 64, 1557-1565). It was likewise possible to show that these EP₂ knockout mice (Hizaki et al. Proc Natl Acad Sci U.S.A. 1999 Aug. 31; 96(18):10501-10506) show distinctly reduced cumulus expansion and severe subfertility, which is to be regarded as causally connected with diminished reproductive processes such as ovulation and fertilization.

The EP₂ receptor accordingly represents an important target for developing medicaments for controlling female fertility. The existence of the 4 subclasses of the PGE₂ receptor opens up the possibility of targeted development of selectively active compounds. However, to date, scarcely any selective EP₂ receptor ligands which bind to the EP₂ subtypes of the PGE₂ receptor are known, since most known compounds also bind to the other PGE₂ receptor subtypes such as, for example, to the EP₄ receptor.

EP₂ receptor antagonists are described, for example in the application US2005059742 (Jabbour, Medical Research Concil). A method in which an EP₂ and/or an EP₄ antagonist can be employed for the treatment of menorrhagia and dysmenorrhea is claimed. AH6809 is disclosed as antagonist of the EP₂ or EP₄ receptor, but no other specific antagonists and no new compounds are disclosed.

In an earlier application of the same group (EP1467738), EP₂ or EP₄ antagonists are described for the treatment of pathological conditions such as, for example, allergic disorders, Alzheimer's disease, pain, abortion, menstrual complaints, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

Ono Pharmaceutical claims in the application WO03/016254 the preparation of benzene acid or saturated carboxylic acid derivatives which are substituted by aryl or heterocycles, inter alia as PGE₂ receptor antagonists. The disclosed compounds are claimed for the treatment of a large number of disorders, including allergic disorders, Alzheimer's disease, pain, abortion, painful menstruation, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

The application WO04/39807 of Merck Frosst, Canada, discloses the preparation of pyridopyrrolizines and pyridoindolizines. However, these compounds are distinguished by good binding to the PGD₂ receptor, and this receptor represents a different subtype of the prostaglandin receptor.

Naphthalene derivatives as EP₄ receptor ligands are disclosed in application US2004102508 of SmithKline Beecham Corporation. The claimed compounds are used for the treatment or prophylaxis of pain, allergic reactions and neurodegenerative disorders.

EP₄ antagonists (γ-lactams) are claimed in the application WO03/103604 (Applied Research Systems). The compounds bind approximately 60-fold better to the EP₄ than to the EP₂ receptor and are claimed inter alia for the treatment of premature labour, dysmenorrhea, asthma, infertility or fertility impairments. The same company claims in the applications WO03/053923 (substituted pyrrolidines) or WO03/035064 (substituted pyrrolidines) compounds for the treatment of disorders associated with prostaglandins, such as, for example, infertility, hypertension and osteoporosis. The compounds bind to the EP₄- and to the EP₂ receptor subtypes. The application WO03/037433 claims ω-cycloalkyl, 17 heteroaryl prostaglandin derivatives as EP₂ receptor antagonists, in particular for the treatment of elevated intraocular pressure.

The application WO03/064391 (Pfizer Products) describes metabolites of [3-[[N-(4-tert-butylbenzyl)(pyridin-3-ylsulphonyl)amino]methyl]acetic acid which inhibit the binding of [³H] prostaglandin E₂ to the EP₂ receptor. The use of these metabolites for the treatment of osteoporosis is disclosed.

Tani et al. claim in the application US2005124577 8-azaprostaglandin derivatives for the treatment of immunological disorders, allergic disorders, premature labour, abortion, etc. The compounds bind to the EP₂ and to the EP₄ receptor.

European patent application EP 1306087 describes EP₂ receptor agonists which are used for the treatment of erectile dysfunction (Ono Pharmaceuticals). The same class of structures is described in European patent EP 860430 (Ono Pharmaceuticals), and their use for the manufacture of a medicament for the treatment of immunological disorders, asthma and abortion is claimed. WO04/009117 describes EP₂ and EP₄ receptor agonists for the treatment of disorders caused by uterine contraction, for example painful menstruation (Ono Pharmaceuticals).

The applications WO03/74483 and WO03/09872 describe agonists which bind equally to the EP₂ and to the EP₄ receptor (Ono Pharmaceuticals).

Agonists of the EP₂ and of the EP₄ receptors are frequently described in connection with the treatment of osteoporosis (WO99/19300 (Pfizer), US2003/0166631 (Dumont Francis), WO03/77910 (Pfizer), WO03/45371 (Pfizer), WO03/74483 and WO03/09872 (Ono Pharmaceuticals)) and for glaucoma treatment (WO04/37813, WO04/37786, WO04/19938, WO03/103772, WO03/103664, WO03/40123, WO03/47513, WO03/47417 (Merck Frosst Canada)) and U.S. Pat. No. 6,410,591 and U.S. Pat. No. 6,747,037 (Allergan).

The patent application WO04/12656 (Applied Research Systems) claims EP₂ receptor agonists in connection with inflammation.

The patent application WO03/77919 (Merck & Co. Inc.) claims EP₄ receptor agonists for the treatment of fertility.

In the application WO01/32632, Eli Lilly claim the use of 4-substituted pyrimidine derivatives which have antagonistic action on the metabotropic glutamate receptor mGluR₁ for the treatment of disorders connected to the mGluR₁ (for example pain or migraine).

Patent application WO05/67546 (Ambit Biosciences Cooperation) claims pyrrolopyrimidine derivatives as protein kinease modulators and their use for the treatment of disorders which are connected to the “fms-like kinase” FLT3 kinase.

Bicyclic heteroaromatic compounds as tyrosine kinease inhibitors are described in the application WO97/13771 (Glaxo), as is their use for the treatment of disorders connected to abnormal tyrosine kinase activity, for the treatment of malignant tumours, of arterial sclerosis, restenosis or thrombosis.

The application WO07/71456 (Bayer Schering Pharma) discloses carbazoles and fluorenes which bind selectively to the EP₂ receptor in the μM range. However, there is still a need for more effective EP₂ receptor modulators for the regulation of the processes which are ultimately responsible for ovulation, fertilization, nidation and decidualization and thus contribute to the promotion or inhibition of fertility.

It is therefore an object of the invention to provide more effective EP₂ receptor modulators.

This object is achieved by the provision of the compounds of the general formula I,

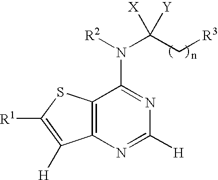

where
R¹ is a CH═CH-aryl group having a 6-10-membered, mono- or bicyclic aryl ring which is in each case unsubstituted or optionally mono- to trisubstituted,
   a CH═CH-heteroaryl group having a 5-10-membered, mono- or bicyclic heteroaryl ring which is in each case unsubstituted or optionally mono- to trisubstituted,
   a 6-10-membered, mono- or bicyclic aryl ring which is in each case unsubstituted or optionally mono- to trisubstituted,
   a 5-10-membered, mono- or bicyclic heteroaryl ring which is in each case unsubstituted or optionally mono- to trisubstituted,
      where the substituents may each be selected from the group of
      halogen, cyano, CH₂CN, R⁴, OR⁴, CH₂OR⁴, OC(O)R⁴, S(O)ₙR⁴ where n=0, 1, 2,
      SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
n is 0, 1 or 2,
X, Y are each independently hydrogen, a methyl group or together are a cyclopropyl ring,
R² is hydrogen, a C₁-C₄-alkyl group,
R³ is a 3-12-membered, mono-, bi- or tricyclic cycloalkyl radical,
   a 6-10-membered, mono- or bicyclic aryl ring which is in each case unsubstituted or optionally mono- to trisubstituted,
   a 5-10-membered, mono- or bicyclic heteroaryl ring which is in each case unsubstituted or optionally mono- to trisubstituted,
   an 8-12-membered aryl-cycloalkyl or heteroaryl-cycloalkyl or aryl-heterocyclyl or heteroaryl-heterocyclyl group which is in each case unsubstituted or optionally mono- to trisubstituted,
   where the substituents may be selected from the group of
      halogen, cyano, CH₂CN, R⁴, OR⁴, CH₂OR⁴, OC(O)R⁴, S(O)ₙR⁴ where n=0, 1, 2,
      SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
R⁴ is hydrogen, a C₁-C₄-alkyl group, a C₂-C₄-alkenyl group, a C₂-C₄-alkynyl group, a C₃-C₆-cycloalkyl group, a CH₂-C₃-C₆-cycloalkyl group, a 6-membered aryl ring, a 5-6-membered heteroaryl ring or a CH₂-aryl or -heteroaryl group, where the aryl radical is 6-membered and the heteroaryl radical is 5- or 6-membered,
R⁵ is hydrogen, a C₁-C₄-alkyl group,
R⁴, R⁵ together are a 3-6-membered ring,
   and their stereoisomers, diastereomers, enantiomers, salts and their cyclodextrin clathrates, and which overcome the known disadvantages, and which have improved properties, i.e. good efficacy, good solubility and stability.

The inventive compounds have an antagonistic action on the EP₂ receptor and thus serve for female fertility control.

The saturated, unbranched C₁-C₄-alkyl substituents specified under R², R⁴ and R⁵ are, for example, a methyl, ethyl, n-propyl, n-butyl group, and the branched C₃-C₄-alkyl groups are an isopropyl, isobutyl, sec-butyl, tert-butyl group.

The alkyl groups may optionally be mono- or polysubstituted by halogen atoms (e.g. fluorine, chlorine or bromine).

The C₂-C₄-alkenyl substituents in R⁴ are each straight-chain or branched, which means, for example, the following radicals: vinyl, allyl, homoallyl, (E)-but-2-enyl, (Z)-but-2-enyl, 2-methylvinyl.

The alkenyl groups may optionally be mono- or polysubstituted by halogen atoms (e.g. fluorine, chlorine or bromine).

The C₂-C₄-alkynyl substituents R⁴ are each straight-chain or branched, which means, for example, the following radicals: ethynyl, prop-1-ynyl, but-1-ynyl, but-2-ynyl.

The alkynyl groups may optionally be substituted by halogen atoms (e.g. fluorine, chlorine or bromine).

Halogen is understood to mean the following: fluorine, chlorine, bromine, iodine.

The 3-12-membered, mono-, bi- or tricyclic cycloalkyl radical specified under R³ comprises monocyclic alkyl rings such as cyclopropyl, cyclobutyl, cyclopentyl, cyclohexyl or cycloheptyl, or cyclooctyl, but also bicyclic rings, for example decahydronaphthalene, tricyclic rings or bridged rings, for example adamantyl, and also heteroatom-containing heterocycles, for example aziridinyl, azetidinyl, pyrrolidinyl, piperidinyl, piperazinyl, morpholinyl, azepanyl, [1,4]-diazepanyl, tetrahydrofuranyl, thiomorpholinyl.

The C₃-C₁₂-cycloalkyl groups are bonded via one of the substitutable positions and may optionally be mono- to disubstituted by halogen atoms (e.g. fluorine, chlorine or bromine). The nitrogen or sulphur atoms may optionally be oxidized to an N-oxide, S-oxide, S,S-dioxide.

The C₃-C₆-cycloalkyl specified under R⁴ comprises alkyl rings such as cyclopropyl, cyclobutyl, cyclopentyl or cyclohexyl, and heteroatom-containing heterocycles, for example aziridinyl, azetidinyl, pyrrolidinyl, tetrahydrofuranyl, piperidinyl, piperazinyl, morpholinyl, thiomorpholinyl.

The C₃-C₆-cycloalkyl groups are bonded via the substitutable positions and may optionally be mono- to disubstituted by halogen atoms (e.g. fluorine, chlorine or bromine). The nitrogen or sulphur atoms may optionally be oxidized to an N-oxide, S-oxide, S,S-dioxide.

The 6-10-membered, mono- or bicyclic aryl radical which is specified in R¹ and R³ may optionally be mono- to trisubstituted is bonded to the skeleton via one of the possible bonding sites.

Examples of a 6-10-membered, mono- or bicyclic aryl radical include the following: phenyl, naphthyl.

The 5-10-membered, mono- or bicyclic heteroaryl radical which is specified in R¹ and R³ and may optionally be mono- to trisubstituted is understood to mean 5-10-membered ring systems which, instead of the carbon, may contain one or more identical or different heteroatoms such as oxygen, nitrogen or sulphur in the ring, may be mono- or bicyclic and are bonded to the skeleton via one of the possible bonding sites. The 5-10-membered, mono- or bicyclic heteroaryl radicals may optionally be mono- to trisubstituted. When the heteroaryl radical is substituted by a hydroxyl group, the corresponding tautomers are included, provided that the hydroxyl group on the heteroaryl radical is capable of this. The nitrogen atoms may optionally be oxidized to an N-oxide.

The 5-10-membered, mono- or bicyclic heteroaryl radicals may be a pyridyl, pyrimidyl, quinolinyl, isoquinolinyl, phthalazinyl, quinazolinyl, quinoxalinyl, cinnolinyl, benzofuranyl, benzothienyl, indolyl, benzimidazolyl, 2,1,3-benzo-thiadiazolyl, 1H-benzotriazolyl, benzothiazolyl, benzoxazolyl, benzisoxazolyl, furanyl, thienyl, oxazolyl, isoxazolyl, isothiazolyl, thiazolyl, isothiazolyl, pyrrolyl, pyrazolyl, pyrazinyl, pyridazinyl, triazinyl, 1H-pyrazolo[3,4-d]pyrimidyl, 1H-indazolyl, triazolyl, oxadiazolyl, tetrazolyl or imidazolyl group bonded via one of the substitutable positions.

The 6-membered aryl radical specified in R⁴ is a phenyl radical which may optionally be mono- to disubstituted by halogen atoms (e.g. fluorine, chlorine or bromine), C₁-C₄-alkyl groups, a cyano group or a hydroxyl group.

The 5-6-membered heteroaryl radical specified in R⁴ is understood to mean 5-6-membered ring systems which, instead of the carbon, may contain one or more, identical or different heteroatoms, such as oxygen, nitrogen or sulphur, in the ring and are bonded to the skeleton via one of the possible bonding sites. The 5-6-membered heteroaryl radicals may optionally be mono- to disubstituted by halogen atoms (e.g. fluorine, chlorine or bromine), C₁-C₄-alkyl groups, a cyano group or a hydroxyl group. When the heteroaryl radical is substituted by a hydroxyl group, the corresponding tautomers are included, provided that the hydroxyl group on the heteroaryl radical is capable of this. The nitrogen atoms may optionally be oxidized to an N-oxide.

The 5-6-membered heteroaryl groups may be a pyridyl, pyrimidyl, furanyl, thienyl, oxazolyl, isoxazolyl, thiazolyl, isothiazolyl, pyrrolyl, pyrazolyl, pyrazinyl, pyridazinyl, triazinyl, triazolyl, oxadiazolyl, tetrazolyl or imidazolyl group bonded via one of the substitutable positions.

The 8-12-membered aryl-cycloalkyl or heteroaryl-cycloalkyl or aryl-heterocyclyl or heteroaryl-heterocyclyl groups specified under R³ are unsubstituted or optionally mono- to trisubstituted and optionally contain, instead of the carbon, one or more identical or different heteroatoms such as oxygen, nitrogen or sulphur in the heteroaryl or heterocyclyl moiety. The nitrogen atoms in the heteroaryl moiety are optionally oxidized to an N-oxide. The oxygen, nitrogen or sulphur atoms in the heterocyclyl moiety are optionally oxidized to an N-oxide, S-oxide, S,S-dioxide. The 8-12-membered aryl-cycloalkyl or heteroaryl-cycloalkyl or aryl-heterocyclyl or heteroaryl-heterocyclyl groups are bonded via one of the substitutable positions and are additionally optionally mono- to disubstituted by an oxo group in the cycloalkyl or heterocyclyl moiety. The 8-12-membered aryl-cycloalkyl or heteroaryl-cycloalkyl or aryl-heterocyclyl or heteroaryl-heterocyclyl groups may optionally be mono- to trisubstituted by halogen atoms (e.g. fluorine, chlorine or bomrine, or C₁-C₄-alkyl groups.

An aryl-cycloalkyl group is, for example, 1,2,3,4-tetrahydronaphthalenyl, indanyl, 3,4-dihydro-2H-naphthalen-1-onyl, indan-1-onyl.

A heteroaryl-cycloalkyl group is, for example, 5,6,7,8-tetrahydroquinolinyl, 5,6,7,8-tetrahydroisoquinolinyl, 5,6,7,8-tetrahydroquinazolinyl, 5,6,7,8-tetrahydroquinoxalinyl, 4,5,6,7-tetrahydro-1H-benzimidazolyl, 4,5,6,7-tetrahydrobenzoxazolyl, 4,5,6,7-tetrahydrobenzthiazolyl, 2,4,5,6-tetrahydrocyclopentapyrazolyl.

An aryl-heterocyclyl group is, for example, 1,2,3,4-tetrahydroquinolinyl, 1,2,3,4-tetrahydroisoquinolinyl, 1,2,3,4-tetrahydroquinazolinyl, 1,2,3,4-tetrahydroquinoxalinyl, 1,2,3,4-tetrahydrophthalazinyl, 2,3-dihydro-1H-indolyl, 2,3-dihydrobenzofuranyl, 2,3-dihydro-1H-isoindolyl, benzo[1,3]dioxolyl, 2,3-dihydrobenzoxazolyl, chromanyl, 2,3-dihydrobenzo[1,4]dioxinyl, 2,3-dihydrophthalazine-1,4-dionyl, isoindole-1,3-dionyl, 2-methylisoindole-1,3-dionyl, 2,3-dihydroisoindol-1-onyl.

A heteroaryl-heterocyclyl group is, for example 2,3-dihydro-1H-pyrrol[3,4-b]quinolin-2-yl, 1,2,3,4-tetrahydrobenz[b][1,7]naphthyridin-2-yl, 1,2,3,4-tetrahydrobenz[b][1,6]naphthyridin-2-yl, 1,2,3,4-tetrahydro-9H-pyrido[3,4-b]indol-2-yl, 1,2,3,4-tetrahydro-9H-pyrido[4,3-b]indol-2-yl, 2,3-dihydro-1H-pyrrolo[3,4-b]indol-2-yl, 1H-2,3,4,5-tetrahydroazepino[3,4-b]indol-2-yl, 1H-2,3,4,5-tetrahydroazepino[4,3-b]indol-3-yl, 1H-2,3,4,5-tetrahydroazepino[4,5-b]indol-2-yl, 5,6,7,8-tetrahydro[1,7]naphthyridyl, 1,2,3,4-tetrahydro[2,7]naphthyridyl, 2,3-dihydro[1,4]dioxino[2,3-b]pyridyl, 2,3-dihydro[1,4]dioxino[2,3-b]pyridyl, 3,4-dihydro-2H-1-oxa[4,6]diazanaphthalenyl, 4,5,6,7-tetrahydro-3H-imidazo[4,5-c]pyridyl, 6,7-dihydro[5,8]diazanaphthalenyl, 1,2,3,4-tetrahydro[1,5]naphthyridinyl, 1,2,3,4-tetrahydro[1,6]naphthyridinyl, 1,2,3,4-tetrahydro[1,7]naphthyridinyl, 1,2,3,4-tetrahydro[1,8]naphthyridinyl, 1,2,3,4-tetrahydro[2,6]naphthyridinyl.

The 3-6-membered ring which can be formed by a ring closure of R⁴ and R⁵ may be a cycloalkyl ring or a heteroatom-containing cycle.

Examples of a 3-6-membered cycloalkyl ring include, for example, the following: cyclopropyl, cyclobutyl, cyclopentyl or cyclohexyl.

Examples of a 3-6-membered, heteroatom-containing heterocycle include, for example, the following: aziridinyl, azetidinyl, pyrrolidinyl, piperidinyl, piperazinyl, morpholinyl, thiomorpholinyl. The nitrogen and sulphur atoms may optionally be oxidized to an N-oxide, S-oxide, S,S-dioxide.

Preference is given to the compounds of the general formula I where
R¹ is a CH═CH-aryl group having a 6-10-membered, mono- or bicyclic aryl ring which is in each case unsubstituted or optionally mono- to trisubstituted,
   a CH═CH-heteroaryl group having a 5-10-membered, mono- or bicyclic heteroaryl ring which is in each case unsubstituted or optionally mono- to trisubstituted,
   a 6-10-membered, mono- or bicyclic aryl ring which is in each case unsubstituted or optionally mono- to trisubstituted,
   a 5-10-membered, mono- or bicyclic heteroaryl ring which is in each case unsubstituted or optionally mono- to trisubstituted,
      where the substituents may each be selected from the group of
      halogen, cyano, CH₂CN, R⁴, OR⁴, CH₂OR⁴, OC(O)R⁴, S(O)ₙR⁴ where n=0, 1, 2,
      SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
n is 0, 1 or 2,
X, Y are each independently hydrogen, a methyl group or together are a cyclopropyl ring,
R² is hydrogen, a methyl group,
R³ is a 3-12-membered, mono-, bi- or tricyclic cycloalkyl radical,
   a 6-10-membered, mono- or bicyclic aryl ring which is in each case unsubstituted or optionally mono- to trisubstituted,
   a 5-10-membered, mono- or bicyclic heteroaryl ring which is in each case unsubstituted or optionally mono- to trisubstituted,
   an 8-12-membered aryl-cycloalkyl or heteroaryl-cycloalkyl or aryl-heterocyclyl or heteroaryl-heterocyclyl group which is in each case unsubstituted or optionally mono- to trisubstituted,
   where the substituents may be selected from the group of
      halogen, cyano, CH₂CN, R⁴, OR⁴, CH₂OR⁴, OC(O)R⁴, S(O)ₙR⁴ where n=0, 1, 2,
      SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
R⁴ is hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or -heteroaryl group, where the aryl radical is 6-membered and the heteroaryl radical is 5- or 6-membered,
R⁵ is hydrogen, a C₁-C₄-alkyl group,
R⁴, R⁵ together form a 3-6-membered ring.

Likewise preferred are the compounds of the general formula I where
R¹ is a CH═CH-aryl group having a 6-10-membered, mono- or bicyclic aryl ring which is in each case unsubstituted or optionally mono- to trisubstituted,
   a CH═CH-heteroaryl group having a 5-10-membered, mono- or bicyclic heteroaryl ring which is in each case unsubstituted or optionally mono- to trisubstituted,
   a 6-10-membered, mono- or bicyclic aryl ring which is in each case unsubstituted or optionally mono- to trisubstituted,
   a 5-10-membered, mono- or bicyclic heteroaryl ring which is in each case unsubstituted or optionally mono- to trisubstituted,
      where the substituents may each be selected from the group of
      halogen, cyano, CH₂CN, R⁴, OR⁴, CH₂OR⁴, OC(O)R⁴, S(O)ₙR⁴ where n=0, 1, 2,
      SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
n is 0, 1 or 2,
X, Y are each independently hydrogen, a methyl group or together are a cyclopropyl ring,
R²: is hydrogen, a methyl group,
R³: is 3-6-membered, monocyclic cycloalkyl radical,
   a 6-10-membered, mono- or bicyclic aryl ring which is in each case unsubstituted or optionally mono- to trisubstituted,
   a 5-10-membered, mono- or bicyclic heteroaryl ring which is in each case unsubstituted or optionally mono- to trisubstituted,
   an 8-12-membered aryl-cycloalkyl or heteroaryl-cycloalkyl or aryl-heterocyclyl or heteroaryl-heterocyclyl group which is in each case unsubstituted or optionally mono- to trisubstituted,
   where the substituents may be selected from the group of
      halogen, cyano, CH₂CN, R⁴, OR⁴, CH₂OR⁴, S(O)ₙR⁴ where n=0, 1, 2,
      SO₂NHR⁴, SO₂NHC(O)R⁴, NHSO₂R⁴,
R⁴ is hydrogen, a C₁-C₄-alkyl group, a C₂-C₄-alkenyl group, a C₂-C₄-alkynyl group, a C₃-C₆-cycloalkyl group, a CH₂-C₃-C₆-cycloalkyl group, a 6-membered aryl ring, a 5-6-membered heteroaryl ring or a CH₂-aryl or -heteroaryl group, where the aryl radical is 6-membered and the heteroaryl radical is 5- or 6-membered,
R⁵ is hydrogen, a C₁-C₄-alkyl group,
R⁴, R⁵ together form a 3-6-membered ring.

According to the present invention, the following compounds are very particularly preferred:
1. (6-Phenylthieno[3,2-d]pyrimidin-4-yl)thiophen-2-ylmethylamine
2. Cyclohexylmethyl-(6-phenylthieno[3,2-d]pyrimidin-4-yl)amine
3. (3-Morpholin-4-ylpropyl)-(6-phenylthieno[3,2-d]pyrimidin-4-yl)amine
4. Furan-2-ylmethyl-(6-phenylthieno[3,2-d]pyrimidin-4-yl)amine
5. (6-Phenylthieno[3,2-d]pyrimidin-4-yl)-(3-pyrrolidin-1-yl-propyl)amine
6. Phenethyl-(6-phenylthieno[3,2-d]pyrimidin-4-yl)amine
7. (6-Phenylthieno[3,2-d]pyrimidin-4-yl)pyridin-4-ylmethylamine
8. (2-Imidazol-1-yl-ethyl)-(6-phenylthieno[3,2-d]pyrimidin-4-yl)amine
9. 4-[2-(6-Phenylthieno[3,2-d]pyrimidin-4-ylamino)ethyl]phenol
10. Benzylmethyl-(6-phenylthieno[3,2-d]pyrimidin-4-yl)amine
11. Methyl-(6-phenylthieno[3,2-d]pyrimidin-4-yl)-(3,4,5-trimethoxybenzyl)amine
12. (6-Phenylthieno[3,2-d]pyrimidin-4-yl)-(tetrahydro-furan-2-ylmethyl)amine
13. (4-Fluorobenzyl)-(6-phenylthieno[3,2-d]pyrimidin-4-yl)amine
14. (2-Chloro-6-fluorobenzyl)-(6-phenylthieno[3,2-d]pyrimidin-4-yl)amine
15. (6-Phenylthieno[3,2-d]pyrimidin-4-yl)-(2-thiophen-2-yl-ethyl)amine
16. (6-Phenylthieno[3,2-d]pyrimidin-4-yl)-(2-pyrrolidin-1-yl-ethyl)amine
17. (6-Phenylthieno[3,2-d]pyrimidin-4-yl)-(2-pyridin-3-yl-ethyl)amine
18. (3-Phenylpropyl)-(6-phenylthieno[3,2-d]pyrimidin-4-yl)amine
19. [2-(4-Fluorophenyl)-ethyl]-(6-phenylthieno[3,2-d]pyrimidin-4-yl)amine
20. 4-[2-(6-Phenylthieno[3,2-d]pyrimidin-4-ylamino)ethyl]benzenesulphonamide
21. (2-Benzoimidazol-1-yl-ethyl)-(6-phenylthieno[3,2-d]pyrimidin-4-yl)amine
22. (3-Imidazol-1-yl-propyl)-(6-phenylthieno[3,2-d]pyrimidin-4-yl)amine
23. [2-(4-Fluorophenyl)-1,1-dimethylethyl]-(6-phenylthieno[3,2-d]pyrimidin-4-yl)amine
24. Benzo[1,3]dioxol-5-ylmethyl-(6-m-tolylthieno[3,2-d]pyrimidin-4-yl)amine
25. Benzo[1,3]dioxol-5-ylmethyl-(6-o-tolylthieno[3,2-d]pyrimidin-4-yl)amine
26. Benzo[1,3]dioxol-5-ylmethyl-[6-(4-fluorophenyl)thieno[3,2-d]pyrimidin-4-yl]amine
27. Benzo[1,3]dioxol-5-ylmethyl-[6-(3-fluorophenyl)thieno[3,2-d]pyrimidin-4-yl]amine
28. Benzo[1,3]dioxol-5-ylmethyl-[6-(4-chlorophenyl)thieno[3,2-d]pyrimidin-4-yl]amine
29. Benzo[1,3]dioxol-5-ylmethyl-[6-(2-fluorophenyl)thieno[3,2-d]pyrimidin-4-yl]amine
30. Benzo[1,3]dioxol-5-ylmethyl-(6-p-tolylthieno[3,2-d]pyrimidin-4-yl)amine
31. Benzo[1,3]dioxol-5-ylmethyl-[6-(3-methoxyphenyl)thieno[3,2-d]pyrimidin-4-yl]-amine
32. Benzo[1,3]dioxol-5-ylmethyl-(6-naphthalen-2-ylthieno[3,2-d]pyrimidin-4-yl)amine
33. Benzo[1,3]dioxol-5-ylmethyl-(6-biphenyl-4-ylthieno[3,2-d]pyrimidin-4-yl)amine
34. Benzo[1,3]dioxol-5-ylmethyl-(6-benzofuran-2-yl-thieno[3,2-d]pyrimidin-4-yl)amine
35. Benzo[1,3]dioxol-5-ylmethyl-[6-(3-chlorophenyl)thieno[3,2-d]pyrimidin-4-yl]amine
36. Benzo[1,3]dioxol-5-ylmethyl-[6-(4-dimethylaminophenyl)thieno[3,2-d]pyrimidin-4-yl]amine
37. (6-Benzo[b]thiophen-2-ylthieno[3,2-d]pyrimidin-4-yl)benzo[1,3]dioxol-5-ylmethylamine
38. Benzo[1,3]dioxol-5-ylmethyl-(6-naphthalen-1-ylthieno[3,2-d]pyrimidin-4-yl)amine
39. Benzo[1,3]dioxol-5-ylmethyl-[6-(3-fluoro-4-methylphenyl)thieno[3,2-d]pyrimidin-4-yl]amine
40. Benzo[1,3]dioxol-5-ylmethyl-[6-(2-fluoro-4-methylphenyl)thieno[3,2-d]pyrimidin-4-yl]amine
41. (4-{4-[(Benzo[1,3]dioxol-5-ylmethyl)amino]thieno[3,2-d]pyrimidin-6-yl}-phenyl)methanol
42. 4-{4-[(Benzo[1,3]dioxol-5-ylmethyl)amino]thieno[3,2-d]pyrimidin-6-yl}benzoic acid
43. Benzo[1,3]dioxol-5-ylmethyl-[6-(3-trifluoromethylphenyl)thieno[3,2-d]pyrimidin-4-yl]amine
44. Benzo[1,3]dioxol-5-ylmethyl-[6-(5-fluoro-2-methoxyphenyl)thieno[3,2-d]pyrimidin-4-yl]amine
45. Benzo[1,3]dioxol-5-ylmethyl-[6-(4-tert-butylphenyl)thieno[3,2-d]pyrimidin-4-yl]amine
46. 1-(3-{4-[(Benzo[1,3]dioxol-5-ylmethyl)amino]thieno[3,2-d]pyrimidin-6-yl}-phenyl)ethanone
47. Benzo[1,3]dioxol-5-ylmethyl-[6-(3,4,5-trifluorophenyl)thieno[3,2-d]pyrimidin-4-yl]amine
48. Benzo[1,3]dioxol-5-ylmethyl-[6-(2,3,4-trimethoxyphenyl)thieno[3,2-d]pyrimidin-4-yl]amine
49. Benzo[1,3]dioxol-5-ylmethyl-[6-(3,4,5-trimethoxyphenyl)thieno[3,2-d]pyrimidin-4-yl]amine
50. Benzo[1,3]dioxol-5-ylmethyl-(6-biphenyl-3-ylthieno[3,2-d]pyrimidin-4-yl)amine
51. Benzo[1,3]dioxol-5-ylmethyl-[6-(3-isopropylphenyl)thieno[3,2-d]pyrimidin-4-yl]amine
52. (3-{4-[(Benzo[1,3]dioxol-5-ylmethyl)amino]thieno[3,2-d]pyrimidin-6-yl}phenyl)methanol
53. Benzo[1,3]dioxol-5-ylmethyl-[6-(3-methylsulphanylphenyl)thieno[3,2-d]pyrimidin-4-yl]amine
54. Benzo[1,3]dioxol-5-ylmethyl-[6-(2-chlorophenyl)thieno[3,2-d]pyrimidin-4-yl]amine
55. (6-Benzo[b]thiophen-3-yl-thieno[3,2-d]pyrimidin-4-yl)benzo[1,3]dioxol-5-yl-methylamine
56. Benzo[1,3]dioxol-5-ylmethyl-[6-((E)styryl)thieno[3,2-d]pyrimidin-4-yl]amine
57. Benzo[1,3]dioxol-5-ylmethyl-(6-quinolin-6-yl-thieno[3,2-d]pyrimidin-4-yl)amine
58. Benzo[1,3]dioxol-5-ylmethyl-[6-(3-pyrrolidin-1-ylphenyl)thieno[3,2-d]pyrimidin-4-yl]amine
59.5-{4-[(Benzo[1,3]dioxol-5-ylmethyl)amino]thieno[3,2-d]pyrimidin-6-yl}-2-fluorobenzonitrile
60. Benzo[1,3]dioxol-5-ylmethyl-[6-(6-fluoro-5-methylpyridin-3-yl)thieno[3,2-d]pyrimidin-4-yl]amine
61. Benzo[1,3]dioxol-5-ylmethyl-[6-(6-methoxypyridin-3-yl)thieno[3,2-d]pyrimidin-4-yl]amine
62. Benzo[1,3]dioxol-5-ylmethyl-[6-(3-chloro-4-methylphenyl)thieno[3,2-d]pyrimidin-4-yl]amine
63. 4-{4-[(Benzo[1,3]dioxol-5-ylmethyl)amino]thieno[3,2-d]pyrimidin-6-yl}-N-methylbenzamide
64. (3-{4-[(Benzo[1,3]dioxol-5-ylmethyl)amino]thieno[3,2-d]pyrimidin-6-yl}phenyl)acetonitrile
65. 2-{4-[(Benzo[1,3]dioxol-5-ylmethyl)amino]thieno[3,2-d]pyrimidin-6-yl}benzamide
66. Benzo[1,3]dioxol-5-ylmethyl-[6-(3-fluoro-4-methoxyphenyl)thieno[3,2-d]pyrimidin-4-yl]amine
67. Benzo[1,3]dioxol-5-ylmethyl-[6-(3-chloro-4-fluorophenyl)thieno[3,2-d]pyrimidin-4-yl]amine
68. N-(3-{4-[(Benzo[1,3]dioxol-5-ylmethyl)amino]thieno[3,2-d]pyrimidin-6-yl}phenyl)methanesulphonamide
69. N-(3-{4-[(Benzo[1,3]dioxol-5-ylmethyl)amino]thieno[3,2-d]pyrimidin-6-yl}phenyl)acetamide
70. Benzo[1,3]dioxol-5-ylmethyl-[6-(5-chlorothiophen-2-yl)thieno[3,2-d]pyrimidin-4-yl]amine
71. Benzo[1,3]dioxol-5-ylmethyl-[6-(3,4-difluorophenyl)thieno[3,2-d]pyrimidin-4-yl]amine
72. Benzo[1,3]dioxol-5-ylmethyl-[6-(4-methoxy-3,5-dimethylphenyl)thieno[3,2-d]pyrimidin-4-yl]amine
73. Benzo[1,3]dioxol-5-ylmethyl-[6-(3,5-difluorophenyl)thieno[3,2-d]pyrimidin-4-yl]amine
74. Benzo[1,3]dioxol-5-ylmethyl-[6-(2,3-difluorophenyl)thieno[3,2-d]pyrimidin-4-yl]amine
75. Benzo[1,3]dioxol-5-ylmethyl-[6-(2,5-difluorophenyl)thieno[3,2-d]pyrimidin-4-yl]amine
76. Benzo[1,3]dioxol-5-ylmethyl-{6-[(E)-2-(4-fluorophenyl)vinyl]thieno[3,2-d]pyrimidin-4-yl}amine
77. Benzo[1,3]dioxol-5-ylmethyl-[6-(2-fluoro-3-methoxyphenyl)thieno[3,2-d]pyrimidin-4-yl]amine
78. (5-{4-[(Benzo[1,3]dioxol-5-ylmethyl)amino]thieno[3,2-d]pyrimidin-6-yl}thiophen-2-yl)methanol
79. Benzo[1,3]dioxol-5-ylmethyl-[6-(3-cyclopropylmethoxyphenyl)thieno[3,2-d]pyrimidin-4-yl]amine
80. Benzo[1,3]dioxol-5-ylmethyl-[6-(3-vinylphenyl)thieno[3,2-d]pyrimidin-4-yl]amine
81. Benzo[1,3]dioxol-5-ylmethyl-[6-(3,5-dimethylphenyl)thieno[3,2-d]pyrimidin-4-yl]amine
82. Benzo[1,3]dioxol-5-ylmethyl-(6-quinolin-3-ylthieno[3,2-d]pyrimidin-4-yl)amine
83. (4-Methoxybenzyl)-[6-(3-methoxyphenyl)thieno[3,2-d]pyrimidin-4-yl]amine
84. (4-Methoxybenzyl)-(6-m-tolylthieno[3,2-d]pyrimidin-4-yl)amine
85. [6-(4-Fluorophenyl)thieno[3,2-d]pyrimidin-4-yl]-(4-methoxybenzyl)amine
86. {3-[4-(4-Methoxybenzylamino)thieno[3,2-d]pyrimidin-6-yl]-phenyl}methanol
87.4-[4-(4-Methoxybenzylamino)thieno[3,2-d]pyrimidin-6-yl]-N-methylbenzamide
88. (4-Methoxybenzyl)-(6-o-tolylthieno[3,2-d]pyrimidin-4-yl)amine
89. N-{3-[4-(4-Methoxybenzylamino)thieno[3,2-d]pyrimidin-6-yl]phenyl}-methanesulphonamide
90. N-{3-[4-(4-Methoxybenzylamino)thieno[3,2-d]pyrimidin-6-yl]phenyl}acetamide
91. 4-{2-[6-(3-Methoxyphenyl)thieno[3,2-d]pyrimidin-4-ylamino]ethyl}phenol
92. 4-[2-(6-m-Tolylthieno[3,2-d]pyrimidin-4-ylamino)ethyl]phenol
93. 4-{2-[6-(3-Hydroxymethylphenyl)thieno[3,2-d]pyrimidin-4-ylamino]ethyl}-phenol
94. 4-[2-(6-o-Tolylthieno[3,2-d]pyrimidin-4-ylamino)ethyl]phenol
95. N-(3-{4-[2-(4-Hydroxyphenyl)ethylamino]thieno[3,2-d]pyrimidin-6-yl}phenyl)acetamide
96. 3-{4-[2-(4-Hydroxyphenyl)ethylamino]thieno[3,2-d]pyrimidin-6-yl}benzoic acid
97. (4-Methylbenzyl)-(6-m-tolylthieno[3,2-d]pyrimidin-4-yl)amine
98. [6-(4-Fluorophenyl)thieno[3,2-d]pyrimidin-4-yl]-(4-methylbenzyl)amine
99. {3-[4-(4-Methylbenzylamino)thieno[3,2-d]pyrimidin-6-yl]phenyl}methanol
100. N-{3-[4-(4-Methylbenzylamino)thieno[3,2-d]pyrimidin-6-yl]phenyl}methanesulphonamide
101. 3-[4-(4-Methylbenzylamino)thieno[3,2-d]pyrimidin-6-yl]benzoic acid
102. (6-Phenylthieno[3,2-d]pyrimidin-4-yl)-(3,4,5-trimethoxybenzyl)amine
103. (2-Methoxybenzyl)-(6-phenylthieno[3,2-d]pyrimidin-4-yl)amine
104. (4-Methoxybenzyl)-(6-phenylthieno[3,2-d]pyrimidin-4-yl)amine
105. (4-Methylbenzyl)-(6-phenylthieno[3,2-d]pyrimidin-4-yl)amine
106. Benzo[1,3]dioxol-5-ylmethyl-[6-(4-methoxyphenyl)thieno[3,2-d]pyrimidin-4-yl]amine.

The present invention provides medicaments which comprise at least one of the compounds of the formula I.

The present invention likewise provides medicaments which comprise the inventive compounds with suitable formulation and carrier substances.

Compared with known prostaglandin E₂ ligands, the novel EP₂ agonists and antagonists are distinguished by greater selectivity and stability.

The present invention provides for the use of the inventive compounds for the production of medicaments which comprise at least one of the compounds of the formula I.

The present invention likewise provides medicaments which comprise the inventive compounds with suitable formulation substances and carriers.

Compared with known prostaglandin E₂ ligands, the novel EP₂ agonists and antagonists are notable for greater selectivity and stability.

The present invention provides medicaments for the treatment and prophylaxis of disorders which include fertility disorders, infectious disorders, cancer, viral infections, cardiovascular disorders, elevated intraocular pressure, glaucoma, skeletal system disorders, angiogenetic disorders, uterine contraction impairments, pain, neuroinflammatory disorders, immunomodulatory infections and nephrological disorders.

Fertility disorders mean the disorders which lead to no ovulation taking place, no fertilization taking place, that the blastocyte development is impaired, that no nidation of a fertilized oocyte occurs and no decidualization takes place, infectious disorders mean disorders caused by unicellular parasites, cancer means solid tumours and leukaemia, viral infections mean for example cytomegalus infections, hepatitis, hepatitis B and C and HIV disorders, immunomodulatory infections mean for example avian influenza, cardiovascular disorders mean ischemic reperfusion disorder, stenoses, arterioscleroses and restenoses, angiogenetic disorders mean for example endometriosis and fibrosis, elevated intraocular pressure means glaucoma, uterine contraction impairments mean for example menstrual complaints, skeletal system disorders mean osteoporosis, neuroinflammatory disorders mean multiple sclerosis, Alzheimer's disease, pain and nephrological disorders mean glomerulonephritis.

The present invention likewise provides medicaments for the treatment and prophylaxis of the disorders detailed above, which comprise at least one compound of the general formula I, and medicaments with suitable formulating substances and carriers.

For the use of the inventive compounds as medicaments, they are converted to the form of a pharmaceutical product which, as well as the active ingredient, comprises inert organic or inorganic pharmaceutical carrier materials which are suitable for enteral or parenteral administration, such as, for example, water, gelatin, gum arabic, lactose, starch, magnesium stearate, talc, vegetable oils, polyalkylene glycols etc. The pharmaceutical products may be in solid form, for example as tablets, coated tablets, suppositories, capsules, in semisolid form, for example as ointments, creams, gels, suppositories, emulsions or in liquid form, for example as solutions, suspensions or emulsions.

They comprise where appropriate excipients which are intended to act for example as fillers, binders, disintegrants, lubricants, solvents, solubilizers, masking flavours, colorant, emulsifiers. Examples of types of excipients for the purpose of the invention are saccharides (mono-, di-, tri-, oligo-, and/or polysaccharides), fats, waxes, oils, hydrocarbons, anionic, nonionic, cationic natural, synthetic or semisynthetic surfactants. They additionally comprise where appropriate excipients such as preservatives, stabilizers, wetting agents or emulsifiers; salts to modify the osmotic pressure or buffers.

The present invention likewise provides these pharmaceutical products.

It is appropriate to produce aerosol solutions for inhalation.

Suitable for oral use are in particular tablets, coated tablets or capsules with talc and/or hydrocarbon carriers or binders, such as, for example, lactose, corn starch or potato starch. Use can also take place in liquid form, such as, for example, as solution to which, where appropriate, a sweetener is added. Clathrates are likewise also suitable for oral use of such compounds, examples of clathrates which may be mentioned being those with alpha-, beta-, gamma-cyclodextrin or else beta-hydroxypropylcyclodextrin.

Sterile, injectable, aqueous or oily solutions are used for parenteral administration. Particularly suitable are injection solutions or suspensions, especially aqueous solutions of active compounds in polyethoxylated castor oil.

Examples suitable and customary for vaginal administration are pessaries, tampons or intrauterine device.

Appropriately prepared crystal suspensions can be used for intraarticular injection.

It is possible to use for intramuscular injection aqueous and oily injection solutions or suspensions and appropriate depot preparations.

For rectal administration, the novel compounds can be used in the form of suppositories, capsules, solutions (e.g. in the form of enemas) and ointments both for systemic and for local therapy.

The novel compounds can be used in the form of aerosols and inhalations for pulmonary administration.

For local use on the eyes, external auditory canal, middle ear, nasal cavity and paranasal sinuses, the novel compounds can be used as drops, ointments and tinctures in appropriate pharmaceutical preparations.

Formulations possible for topical application are gels, ointments, fatty ointments, creams, pastes, dusting powders, milk and tinctures. The dosage of the compounds of the general formula I should in these preparations be 0.01%-20% in order to achieve an adequate pharmacological effect.

The dosage of the active ingredients may vary depending on the route of administration, age and weight of the patient, nature and severity of the disorder to be treated and similar factors. Treatment can take place by single dosages or by a large number of dosages over a prolonged period. The daily dose is 0.5-1000 mg, preferably 50-200 mg, it being possible to give the dose as a single dose to be administered once or divided into 2 or more daily doses.

Carrier systems which can be used are also surface-active excipients such as salts of bile acids or animal or vegetable phospholipids, but also mixtures thereof, and liposomes or constituents thereof.

The present invention likewise provides the formulations and dosage forms described above.

Administration of the compounds of the invention can take place by any conventional method, including oral and parenteral, e.g. by subcutaneous or intramuscular injections. The present invention likewise provides enteral, parenteral, vaginal and oral administrations.

The compounds of the invention of the general formula I bind to the EP₂ receptor and have agonistic or antagonistic effect. It is possible to determine whether an agonistic or an antagonistic effect is present by an agonism test (see Example 1.2.1. of the Biological Examples) or by an antagonism test (see Example 1.2.2. of the Biological Examples).

Antagonists mean molecules which bind to their corresponding receptors and which inhibit the initiation of the signal transduction pathway(s) coupled to the receptor by the naturally occurring ligand(s). The antagonists normally compete with the naturally occurring ligand of the receptor for binding to the receptor. However, other modifications of the receptor are also possible by molecules which prevent the signal transduction pathways coupled to the receptor being activated by the naturally occurring ligand(s) (e.g. non-competitive, steric modifications of the receptor).

Receptor antagonists typically bind selectively to their particular receptor and not to other receptors. They normally have a higher binding affinity than the natural ligand. Although antagonists which have a higher affinity for the receptor than the natural ligand are preferred, it is likewise possible to employ antagonists having a lower affinity. However, other modifications of the receptor are also possible by molecules which prevent the signal transduction pathways coupled to the receptor being activated by the naturally occurring ligand(s) (e.g. non-competitive, steric modifications of the receptor).

The antagonists preferably bind reversibly to their corresponding receptors.

The EP₂ receptor antagonist has a preferred affinity for the EP₂ receptor compared with any other EP receptor. The antagonism is measured in the presence of the natural agonist (PGE₂).

Agonists mean molecules which bind to their corresponding receptors and normally compete with the naturally occurring ligand of the receptor for binding to the receptor, and which stimulate the initiation of the signal transduction pathway coupled to the receptor. Agonists may also assist the binding of the natural ligand.

Receptor agonists typically bind selectively to their particular receptor and not to other receptors. They normally have a higher binding affinity than the natural ligand. Although agonists which have a higher affinity for the receptor than the natural ligand are preferred, it is likewise possible to employ agonists having a lower affinity.

The agonists preferably bind reversibly to their corresponding receptors.

The EP₂ receptor agonist has a preferred affinity for the EP₂ receptor compared with any other EP receptor.

Agonists are tested via the initiation of the signal transduction and/or physiological effect mediated by the corresponding receptor.

The compounds or low molecular weight substances which bind to a receptor are referred to as ligands. Their binding is normally reversible. Binding of a ligand to the corresponding receptor activates or inactivates the signal transduction pathway coupled to the receptor. The ligand mediates its intracellular effect in this manner. Ligands mean agonists and antagonists of a receptor.

The substance of Example 69 shows no inhibition in the cellular agonism test but a good activity (IC₅₀=0.46×10 E−6 M) in the antagonism test.

The present invention likewise provides for the use of the substances of the invention as EP₂ receptor antagonists for the treatment of disorders which are caused by disturbances in the signal transduction chain in which the EP₂ receptor is involved, such as, for example, pain and fertility disorders, and which are likewise suitable for controlling fertility.

The oocyte is surrounded in the preovulatory antral follicle by cumulus cells which form a dense ring of cells around the oocyte. After the lutenizing hormone peak (LH peak), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140(2):307-317). This cumulus expansion is an important constituent of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96(18):10501-6.) show a distinctly reduced cumulus expansion and severe subfertility, demonstrating the importance of the prostanoid EP₂ receptor for this process.

The substances of the invention have inhibitory effects in cumulus expansion tests.

The present invention provides for the use of the substances of the invention for controlling fertility.

While the EP₂ receptor antagonist AH 6809 suppresses the expansion of the cumulus by only approx. 30% only at a concentration of 100-200 M, it is possible to achieve a 45% suppression of cumulus expansion in the presence of the substance of example 9, even at a concentration lower by 10-20-fold (10 μM). In these tests, the test substances compete with the natural EP₂ receptor agonist PGE₂.

The present invention provides for the use of the substances of the invention for inhibiting cumulus expansion and thus ovulation and fertilization for contraception.

Prostaglandins play an important part in angiogenesis (Sales, Jabbour, 2003, Reproduction 126, 559-567; Kuwano et al., 2004, FASEB J. 18, 300-310; Kamiyama et al., 2006, Oncogene 25, 7019-7028; Chang et al. 2005, Prostaglandins & other Lipid Mediators 76, 48-58).

Endometriosis is a chronic disorder caused by impairments of blood vessels. About 10% of women regularly suffer from heavy bleeding during menstruation, caused by changes in the blood vessels of the endometrium. In addition, structural differences in the blood vessels have been observed, such as, for example, incomplete formation of the smooth muscle cell layer (Abberton et al., 1999, Hum. Reprod. 14, 1072-1079). Since the blood loss during menstruation is partly controlled by constriction of the blood vessels, it is obvious that the defects in the smooth muscles make a substantial contribution to the bleeding.

The present invention provides for the use of the substances of the general formula I for treating endometriosis.

Prostaglandins play an important part in uterine contraction, and excessively strong contractions are responsible for menstrual complaints (Sales, Jabbour, 2003, Reproduction 126, 559-567).

The present invention provides for the use of the substances of the general formula I for the treatment of menstrual complaints.

Increasing research results also demonstrate the importance of EP receptors, and especially of the EP₂ receptor, in a large number of types of cancer (e.g. breast cancer, colon carcinoma, lung cancer, prostate cancer, leukaemia, skin cancer), suggesting future possibilities of employing modulators (antagonists or agonists) of the EP2 receptor for the therapy and prevention (prophylactic and/or adjuvant) of cancer (Fulton et al. Cancer Res 2006; 66(20): 9794-7; Castellone et al. Science VOL 310 2005, 1504-1510; Chang et al. Cancer Res 2005; 65(11): 4496-9); Hull et al. Mol Cancer Ther 2004; 3(8):1031-9; Richards et al. J Clin Endocrinol Metab 88: 2810-2816, 2003; Sinha et al. 2007, Cancer Res; 67(9):4507-13; Wang et al. 2004, Seminars in Oncology, Vol 31, No 1, Suppl 3: pp 64-73), Jain et al. Cancer Res 2006; 66(13): 6638-48)).

The present invention provides for the use of the substances of the general formula I for the treatment and prevention of cancers.

Prostaglandins also play an important part in processes counteracting osteoporosis. The present invention therefore provides for the use of the substances of the invention for the treatment of osteoporosis.

Reinold et al. (J. Clin. Invest. 115, 673-679 (2005)) describes PGE₂ receptors of the EP₂ subtype as the key signaling elements in inflammatory hyperalgesia. Mice no longer having this receptor (EP₂^{-/-}) do not experience spinal inflammatory pain. There is evidence that an inflammatory, increased pain sensitivity can be treated by targeted modulation of EP₂ receptors.

The present invention provides for the use of the substances of the invention for the treatment of inflammatory hyperalgesia.

Prostaglandins are important mediators of inflammatory processes. Recent research results show the involvement of the EP2 receptor in inflammatory bowel diseases (e.g. Crohn's disease): Sheibanie et al. The Journal of Immunology, 2007, 178: 8138-8147.

The present invention provides for the use of the substances of the invention for the treatment of inflammatory disorders, for example inflammatory bowel diseases, such as Crohn's disease.

The invention also relates to a process for preparing the inventive compounds of the general formula I, which is characterized in that a compound of the general formula Va or Vb,

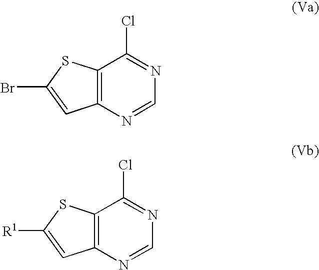

in which R¹ in compound Vb is as defined above is reacted with an amine of the general formula III,

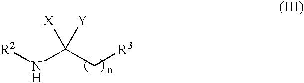

in which X, Y, n, R² and R³ are each as defined above by the methods known to those skilled in the art to give the inventive compounds of the general formula I or the intermediates of the formula II.

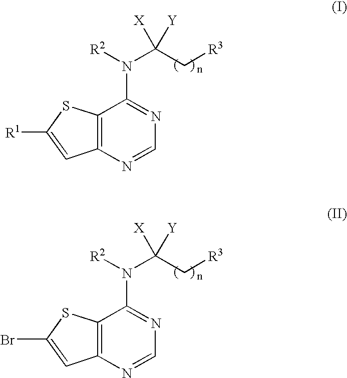

The intermediates of the general formula II are converted by the reaction with a boronic acid or a boronic ester R¹B(OH)₂ of the general formula IV in which R¹ is as defined above in a palladium(0)-catalysed reaction by methods known to those skilled in the art to give further inventive compounds of the general formula I.

The chlorothienopyrimidine of the general formula Va or Vb can be reacted with an amine of the general formula III in an inert solvent or solvent mixture, for example N,N-dimethylformamide, N,N-dimethylacetamide, toluene, n-butanol, tetrahydrofuran, NMP, optionally with addition of an auxiliary base, for example N,N-dimethylaminopyridine, diisopropylethylamine, triethylamine, at temperatures between +20° C. and +165° C., preferably at from +60° C. to +120° C.

A further possibility consists in performing the reaction of the chlorothienopyrimidine of the general formula Va or Vb with an amine of the general formula III in an inert solvent or solvent mixture, for example N-methylpyrrolidinone, toluene, under palladium(0) catalysis (with, for example, Pd(OAc)₂, Pd(PPh₃)₄, Pd₂(dba)₃, PdCl₂(dppf)) and with addition of a base, for example sodium tert-butoxide, and of a suitable ligand, for example 2, 2′-bis(diphenylphosphino)-1,1′-binaphthyl, at temperatures between +40° C. and +150° C.

The compounds of the general formula II obtained from the above-described reaction of the thienopyrimidine Va with an amine of the general formula III are converted to the compounds of the general formula I, for example, by reaction with a boronic acid or a boronic ester of the general formula IV in a solvent or solvent mixture, for example N-methylpyrrolidinone, toluene, THF, ethanol, n-butanol, water, under palladium(0) catalysis (with, for example, Pd(OAc)₂, Pd(PPh₃)₄, Pd₂(dba)₃, PdCl₂(dppf)) and with addition of a base, for example sodium carbonate, potassium carbonate, triethylamine, and of a suitable ligand, for example triphenylphosphine, tri-o-tolylphosphine, at temperatures between +40° C. and +150° C.

The salts are prepared in a customary manner, by admixing a solution of the compound of the formula I with the equivalent amount or an excess of a base or acid, which is optionally in solution, and removing the precipitate or working the solution up in a customary manner.

The invention thus also relates to medicaments based on compounds of the general formula I and on the customary assistants or carriers.

Where the preparation of the starting compounds is not described, they are known or can be prepared analogously to known compounds or to processes described here. It is likewise possible to perform all reactions described here in parallel reactors or by means of combinatorial techniques.

The inventive compounds of the general formula I can be prepared as described in the examples.

Proceeding from the compounds of the general formula Va-b, the compounds of the general formula I and II can be prepared by reacting with amines of the general formula III by processes known to those skilled in the art. It is likewise possible to prepare the inventive compounds of the general formula I by reacting compounds of the general formula II with boronic acids or boronic esters of the general formula IV by processes known to those skilled in the art. An analogous procedure using homologous reagents to the reagents described in the examples allows the further compounds of the general formula I to be obtained.

The substituents on the R¹ and R³ radical in the compounds of the general formula I obtained in this way can be converted further by methods known to those skilled in the art to various functional groups and hence further compounds of the general formula I.

For example, a bromide or chloride can be replaced by an aryl or heteroaryl ring, a substituted alkene or alkyne, amine or a cyano group by means of palladium(0)-catalysed reactions.

A carboxyl function, cyano group or an amine can be converted, for example, to esters and amides of the general formula I, for example by methods known to those skilled in the art.

It is likewise possible, for example, to convert ester functions or a cyano group in compounds of the general formula I, after reduction to the aldehyde, by methods known to those skilled in the art, to further olefins or secondary alcohols substituted by alkyl or aryl radicals. It is likewise possible to convert a cyano group in compounds of the general formula I, by methods known to those skilled in the art, to ketones substituted by alkyl or aryl radicals, which can then be reduced to the corresponding secondary alcohols or else, by methods known to those skilled in the art, can be converted to tertiary alcohols substituted by alkyl or aryl radicals.

The compound Va can be prepared, for example, by processes known to those skilled in the art proceeding from compound VIII by cyclization to give compound VIIa, chlorination to give compound VI and subsequent bromination to give compound Va.

The compounds of the general formula Vb can be prepared, for example, proceeding from compounds of the general formula IX by cyclization to give compounds of the general formula VIIb and subsequent chlorination to give compounds of the general formula Vb by processes known to those skilled in the art.

### Frequently Used Abbreviations:

### NMP N-methylpyrrolidinone

M molar
TFA trifluoroacetic acid
ACN acetonitrile
eq. equivalents
THF tetrahydrofuran
AC acetyl

The examples which follow serve to further illustrate the invention:

Synthesis of the Thienopyrimidine Va Preceding from Thiophene VIII

### 3H-Thieno[3,2-d]pyrimidin-4-one VIIa

10.22 g of ammonium formate are added to a solution of 10 g of methyl 3-formylaminothiophene-2-carboxylate VIII in 12.8 ml of formamide and the mixture is stirred at 140° C. for 10 hours. Subsequently, the mixture is cooled to 25° C., water is added and the precipitate formed is filtered off with suction, washed with water and dried under reduced pressure. In this way, 5.74 g of compound VIIa are obtained.

NMR (300 MHz, DMSO-d₆): δ=7.36 (1H), 8.11 (1H), 8.14 (1H), 12.43 (1H).

### 4-Chlorothieno[3,2-d]pyrimidine VI

To a solution of 6.12 ml of dimethylformamide in 45 ml of methylene chloride is added dropwise at 25° C. a solution of 9.95 ml of oxalyl chloride in 45 ml of methylene chloride. Subsequently, 5.5 g of compound VIIa are added and then the mixture is heated at reflux for 2.5 hours. The reaction mixture is added cautiously to water and extracted three times with methylene chloride. The combined organic phases are dried over sodium sulphate and, after filtration, concentrated under reduced pressure. After drying under reduced pressure, 4.9 g of compound VI are obtained as a crude product, which is reacted further without further purification.

NMR (300 MHz, DMSO-d6): δ=7.73 (1H), 8.57 (1H), 9.01 (1H).

### 6-Bromo-4-chlorothieno[3,2-d]pyrimidine Va

To a solution of 10.32 ml of lithium diisopropylamide (2M in heptane/tetrahydrofuran/ethylbenzene) in 35 ml of THF are slowly added dropwise, at −78° C., 2.95 g of compound VI dissolved in 20 ml of THF and the mixture is stirred at this temperature for 20 minutes. 2.26 ml of 1,2-dibromo-1,1,2,2-tetrafluoroethane are then slowly added dropwise, and the mixture is stirred first at −78° C. for 20 minutes and then at 25° C. for a further 2 hours. The reaction mixture is poured into water and extracted three times with methylene chloride. The combined organic phases are dried over sodium sulphate and, after filtration, dried under reduced pressure. The residue thus obtained is purified by column chromatography on silica gel with hexane/0-50% ethyl acetate. In this way, 2.0 g of compound Va are obtained.

NMR (300 MHz, DMSO-d6): δ=8.04 (1H), 8.99 (1H).

Illustrative Synthesis of a Thienopyrimidine of the General Formula Vb Preceding from a Thiophene of the General Formula IX

### 6-Phenyl-3H-thieno[3,2-d]pyrimidin-4-one VIIb

21.4 g of ammonium acetate are added to a solution of 50 g of methyl 3-amino-5-phenylthiophene-2-carboxylate IX in 168 ml of formic acid and the mixture is then heated under reflux for 6 hours. After cooling to 25° C., the mixture is filtered and the crystals obtained are washed with water and then dried under reduced pressure. 62.3 g of the resulting formate are added to 56.2 ml of formamide and admixed with 45.1 g of ammonium formate. The mixture is stirred at 140° C. for 10 hours and cooled to 25° C., water is added and the precipitate formed is filtered off with suction. The precipitate is washed with water and dried under reduced pressure. In this way, 37.2 g of compound VIIb are obtained.

NMR (300 MHz, DMSO-d6): δ=7.38-7.52 (3H), 7.78-7.86 (3H), 8.13 (1H).

### 4-Chloro-6-phenylthieno[3,2-d]pyrimidine Vb

5.0 g of compound VIIb are heated at reflux in 15.3 ml of phosphorous oxychloride for 2 hours. The reaction mixture is added cautiously to ice-water, the pH is adjusted to 10 with dilute sodium hydroxide solution and extraction is effected three times with methylene chloride. The combined organic phases are washed once with water and dried over sodium sulphate and, after filtration, concentrated under reduced pressure. The residue thus obtained is purified by column chromatography on silica gel with hexane/0-40% ethyl acetate. In this way, 3.8 g of compound Vb are obtained.

NMR (300 MHz, DMSO-d6): δ=7.48-7.57 (3H), 7.94-8.00 (2H), 8.21 (1H), 8.99 (1H).

### General Procedure for the Synthesis of the Thienopyrimidines I and II by Aminating the Compounds Va-b

The corresponding chlorine compound Va-b is initially charged in concentration 0.1 M in NMP, 2 eq of triethylamine and 2 eq of the corresponding amine III (c=0.5 M in NMP) are added and the mixture is heated to 170° C. in a microwave with stirring for 30 min. Thereafter, the reaction mixture is concentrated by rotary evaporation under reduced pressure and purified by means of medium-pressure chromatography on silica gel with hexane/0-100% ethyl acetate or preparative HPLC (column: Purospher Star RP C18 4.6×125 5 μm; flow rate 1 ml/min; eluent A: 0.1% TFA in H₂O, B 0.1% TFA in ACN; gradient, based in each case on B: 5% to 95% (10′) to 95% (2′) to 5% (0.5′) to 5% (2.5′), MS: (M+H)⁺).

According to this general reaction method, for example, the following compounds were synthesized: 1-23 & 102-105.

### General Procedure for the Synthesis of the Thienopyrimidines I by Suzuki Coupling of the Compounds II

The corresponding bromine compound II is initially charged in concentration 0.2 M in THF, 1.5 eq of boronic acid or boronic ester IV, 3 eq of triethylamine (0.6 M in THF), 7.5 mol % of Pd(OAc)₂ (0.0375M in THF), 15 mol % of tri-o-tolylphosphine (0.05M in THF) and 100 eq of water are added, and the mixture is heated in a microwave to 120° C. for 30-40 min. Thereafter, the reaction mixture is concentrated by rotary evaporation under reduced pressure and purified by means of medium-pressure chromatography on silica gel with hexane/0-100% ethyl acetate or preparative HPLC (column: Purospher Star RP C18 4.6×125 5 μm; flow rate 1 ml/min; eluent A: 0.1% TFA in H₂O, B 0.1% TFA in ACN; gradient, based in each case on B: 5% to 95% (10′) to 95% (2′) to 5% (0.5′) to 5% (2.5′), MS: (M+H)⁺).

According to this general reaction method, for example, the following compounds were synthesized: 24-101 & 106.

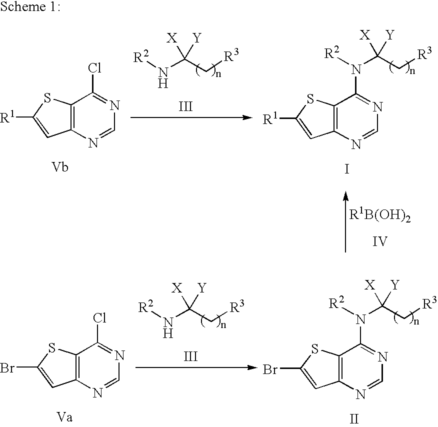

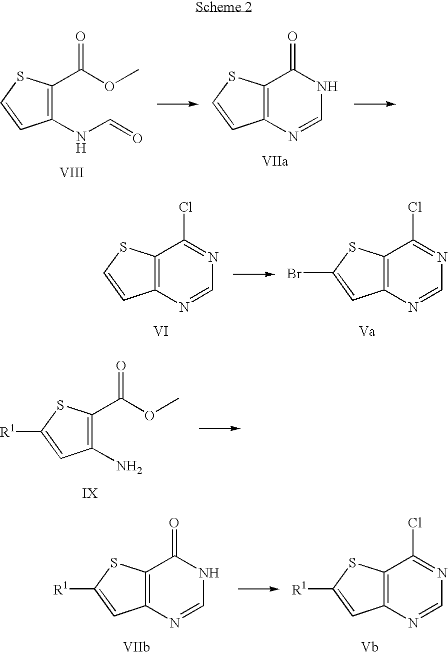

| | | Retention | Mass | Mass |
|---|---|---|---|---|
| Example | Structure | time | calculated | found |
| 1 | 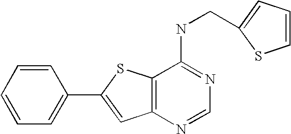 | 6.97 | 323 | 324 |
| 2 | 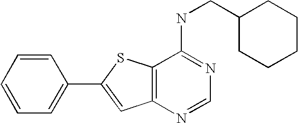 | 7.8 | 323 | 324 |
| 3 | 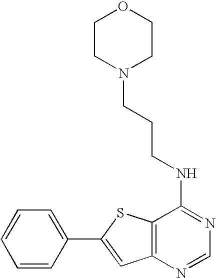 | 4.94 | 354 | 355 |
| 4 | 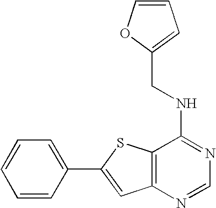 | 6.52 | 307 | 308 |
| 5 | 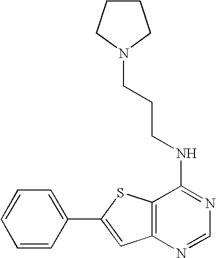 | 4.92 | 338 | 339 |
| 6 | 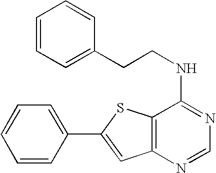 | 7.39 | 331 | 332 |
| 7 | 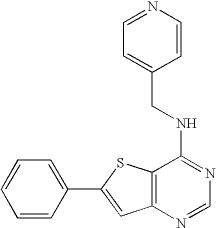 | 4.84 | 318 | 319 |
| 8 | 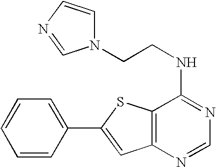 | 4.72 | 321 | 322 |
| 9 | 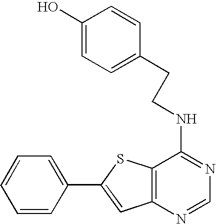 | 6.53 | 347 | 348 |
| 10 | 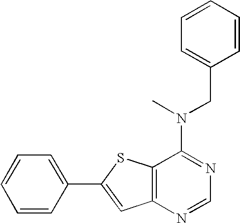 | 7.47 | 331 | 332 |
| 11 | 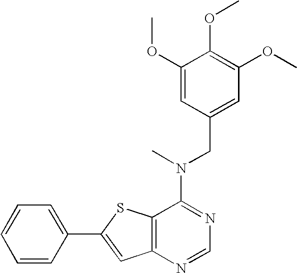 | 7.09 | 421 | 423 |
| 12 | 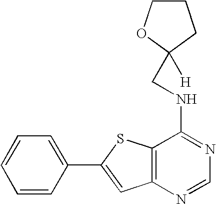 | 6.14 | 311 | 312 |
| 13 | 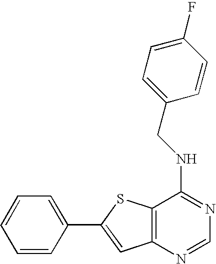 | 7.2 | 335 | 336 |
| 14 | 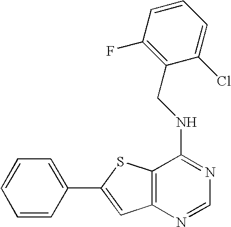 | 7.39 | 369 | 371 |
| 15 | 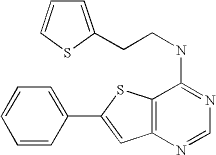 | 7.22 | 337 | 338 |
| 16 | 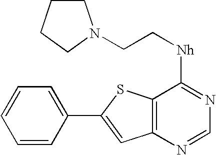 | 4.85 | 324 | 325 |
| 17 | 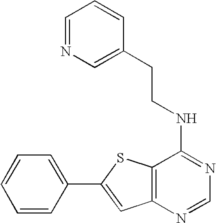 | 4.95 | 332 | 333 |
| 18 | 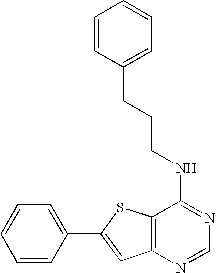 | 7.62 | 345 | 346 |
| 19 | 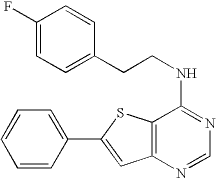 | 7.49 | 349 | 350 |
| 20 | 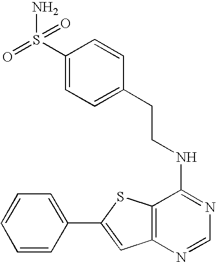 | 6.2 | 410 | 412 |
| 21 | 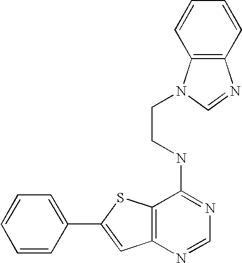 | 5.17 | 371 | 372 |
| 22 | 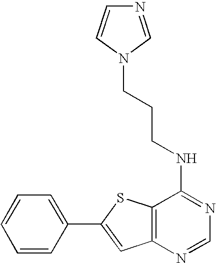 | 4.99 | 335 | 336 |
| 23 | 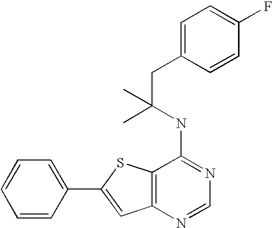 | 8 | 377 | 378 |
| 24 | 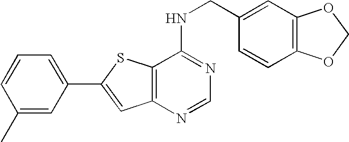 | 7.25 | 375 | 376 |
| 25 | 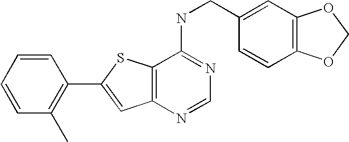 | 7.14 | 375 | 376 |
| 26 | 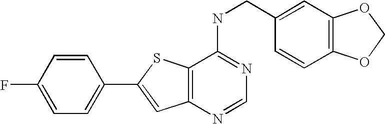 | 6.97 | 379 | 380 |
| 27 | 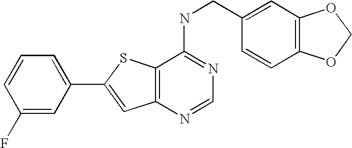 | 7.06 | 379 | 380 |
| 28 | 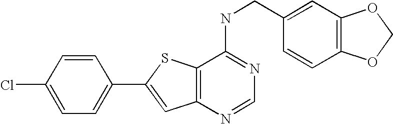 | 7.34 | 395 | 397 |
| 29 | 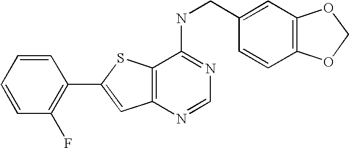 | 6.93 | 379 | 380 |
| 30 | 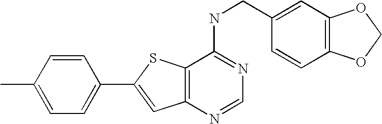 | 7.22 | 375 | 376 |
| 31 | 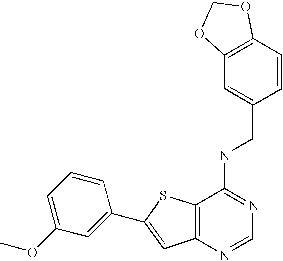 | 7.03 | 391 | 392 |
| 32 | 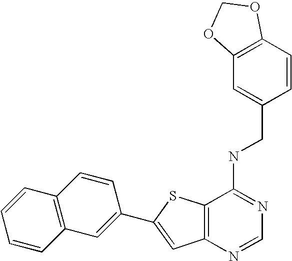 | 7.66 | 411 | 412 |
| 33 | 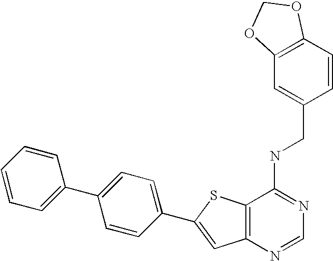 | 7.93 | 437 | 439 |
| 34 | 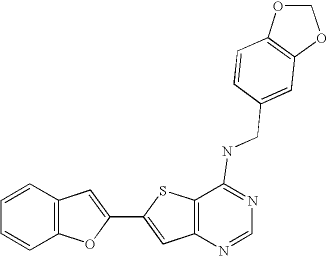 | 7.49 | 401 | 402 |
| 35 | 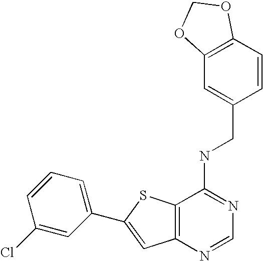 | 7.38 | 395 | 397 |
| 36 | 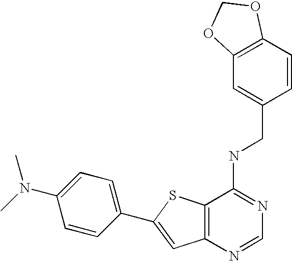 | 7.19 | 404 | 405 |
| 37 | 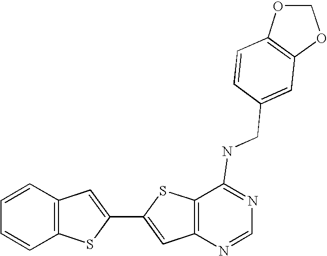 | 7.65 | 417 | 419 |
| 38 | 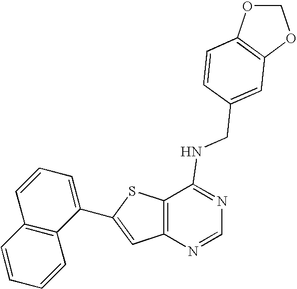 | 7.56 | 411 | 412 |
| 39 | 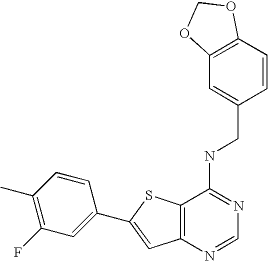 | 7.34 | 393 | 394 |
| 40 | 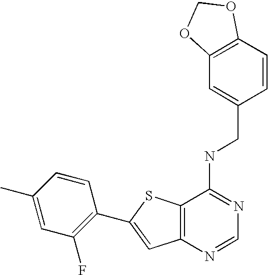 | 7.34 | 393 | 394 |
| 41 | 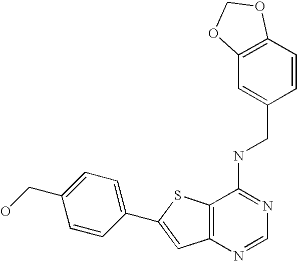 | 6.15 | 391 | 392 |
| 42 | 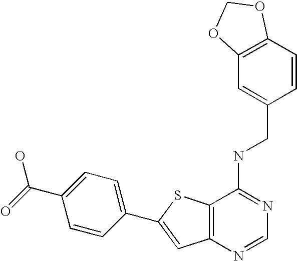 | 6.33 | 405 | 406 |
| 43 | 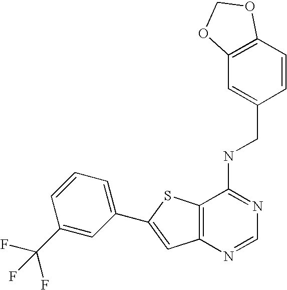 | 7.47 | 429 | 430 |
| 44 | 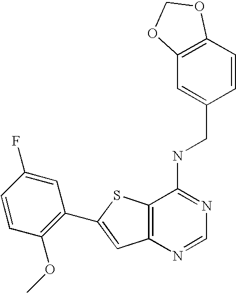 | 7.16 | 409 | 410 |
| 45 | 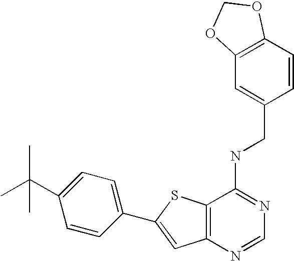 | 8.12 | 417 | 419 |
| 46 | 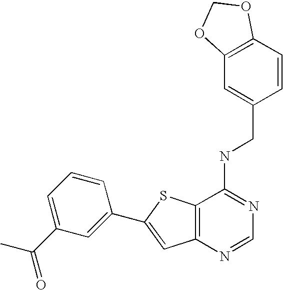 | 6.7 | 403 | 404 |
| 47 | 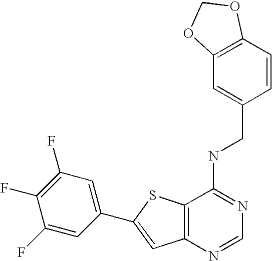 | 7.35 | 415 | 416 |
| 48 | 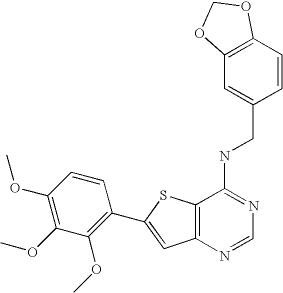 | 7.09 | 451 | 453 |
| 49 | 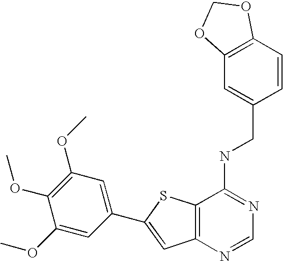 | 6.89 | 451 | 453 |
| 50 | 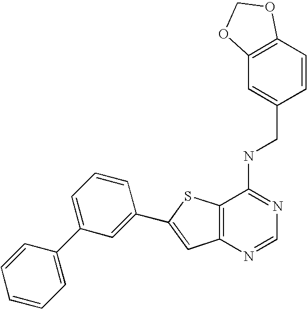 | 8.03 | 437 | 439 |
| 51 | 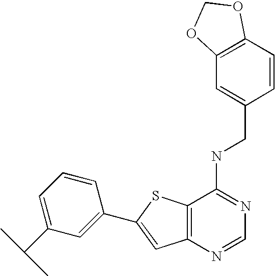 | 7.83 | 403 | 405 |
| 52 | 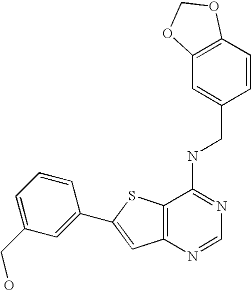 | 6.15 | 391 | 392 |
| 53 | 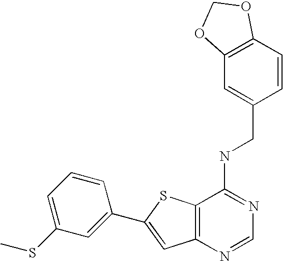 | 7.4 | 407 | 409 |
| 54 | 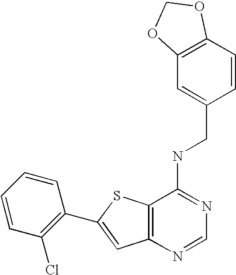 | 7.14 | 395 | 397 |
| 55 | 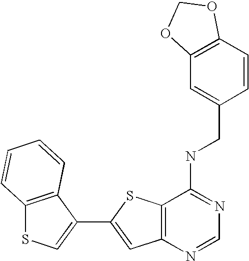 | 7.49 | 417 | 419 |
| 56 | 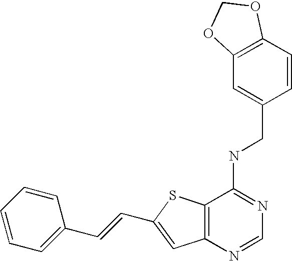 | 7.39 | 387 | 388 |
| 57 | 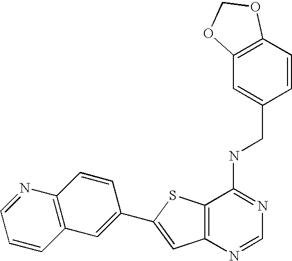 | 5.62 | 412 | 413 |
| 58 | 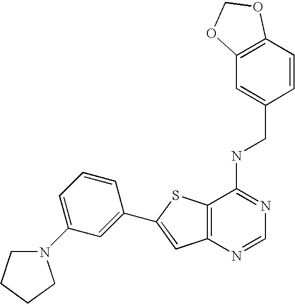 | 7.85 | 430 | 432 |
| 59 | 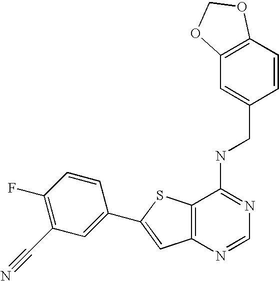 | 6.91 | 404 | 405 |
| 60 | 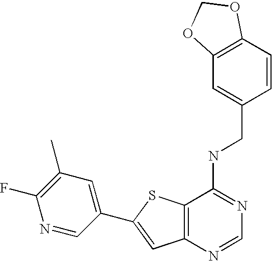 | 6.72 | 394 | 395 |
| 61 | 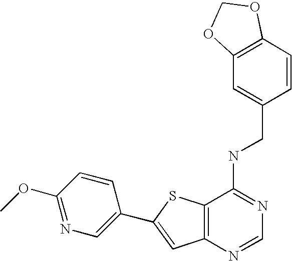 | 6.63 | 392 | 393 |
| 62 | 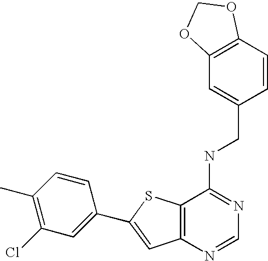 | 7.68 | 409 | 411 |
| 63 | 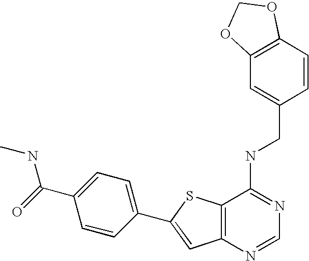 | 6.05 | 418 | 419 |
| 64 | 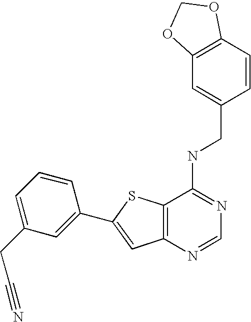 | 6.64 | 400 | 401 |
| 65 | 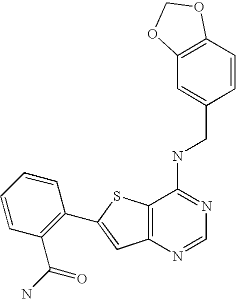 | 5.78 | 404 | 405 |
| 66 | 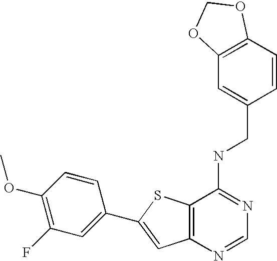 | 7.07 | 409 | 410 |
| 67 | 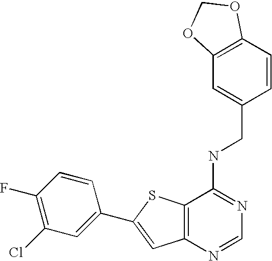 | 7.38 | 413 | 415 |
| 68 | 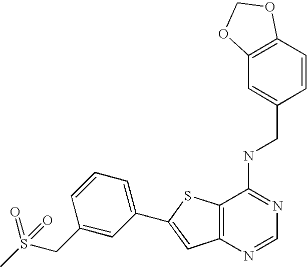 | 6.44 | 454 | 456 |
| 69 | 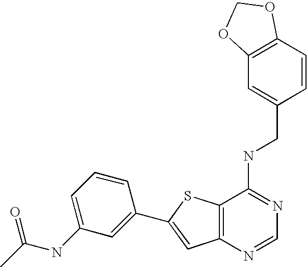 | 6.33 | 418 | 419 |
| 70 | 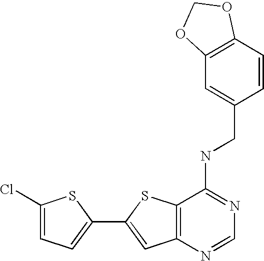 | 7.36 | 401 | 403 |
| 71 | 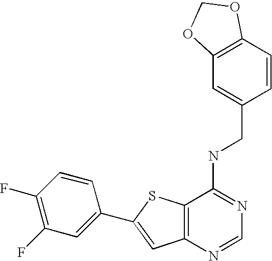 | 7.17 | 397 | 398 |
| 72 | 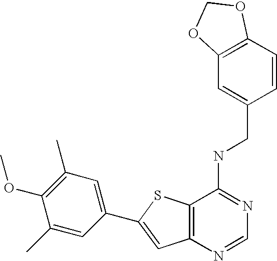 | 7.51 | 419 | 421 |
| 73 | 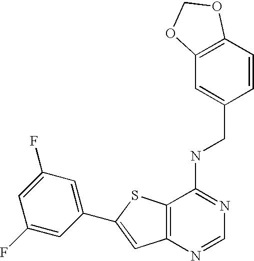 | 7.19 | 397 | 398 |
| 74 | 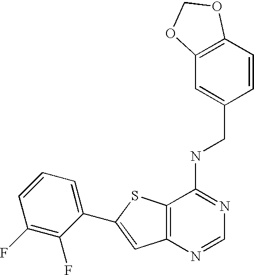 | 7.15 | 397 | 398 |
| 75 | 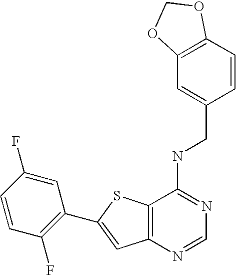 | 7.09 | 397 | 398 |
| 76 | 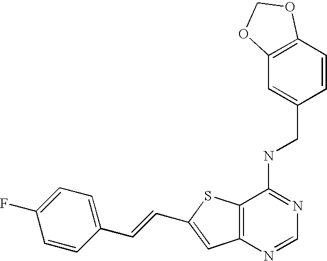 | 7.43 | 405 | 406 |
| 77 | 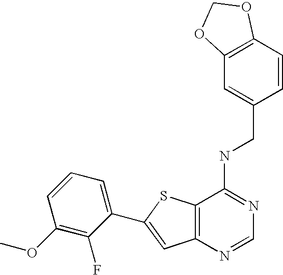 | 7.07 | 409 | 410 |
| 78 | 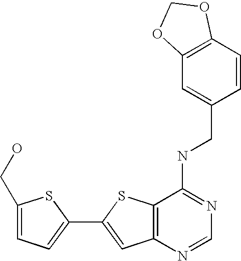 | 6.18 | 397 | 398 |
| 79 | 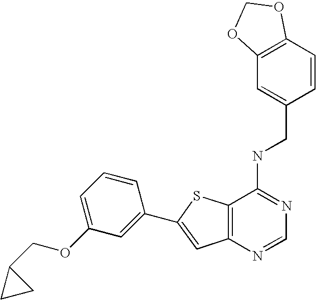 | 7.71 | 431 | 433 |
| 80 | 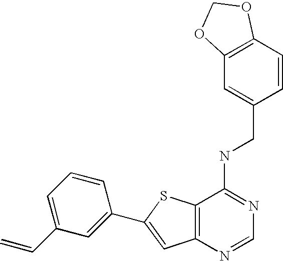 | 7.44 | 387 | 388 |
| 81 | 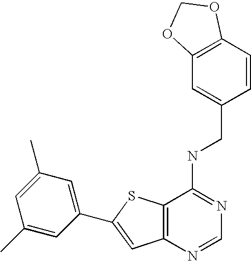 | 7.59 | 389 | 390 |
| 82 | 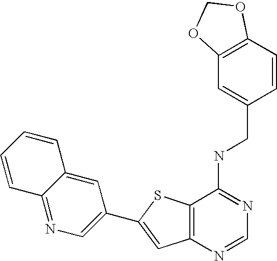 | 6.58 | 412 | 413 |
| 83 | 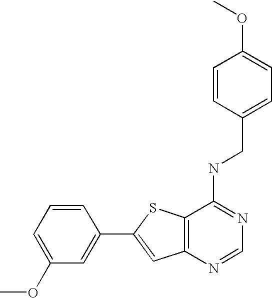 | 3.21 | 377 | 378 |
| 84 | 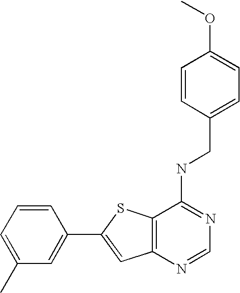 | 3.43 | 361 | 362 |
| 85 | 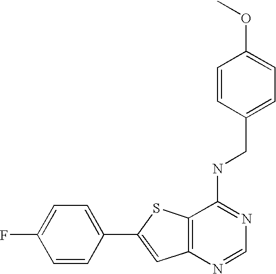 | 3.07 | 365 | 366 |
| 86 | 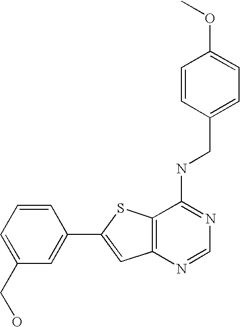 | 2.81 | 377 | 378 |
| 87 | 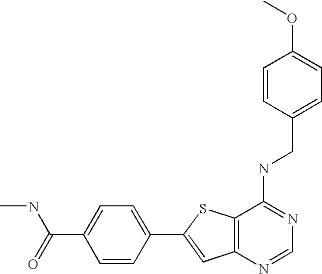 | 2.76 | 404 | 405 |
| 88 | 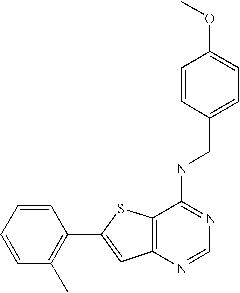 | 3.2 | 361 | 362 |
| 89 | 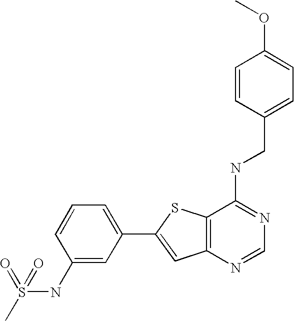 | 2.85 | 441 | 442 |
| 90 | 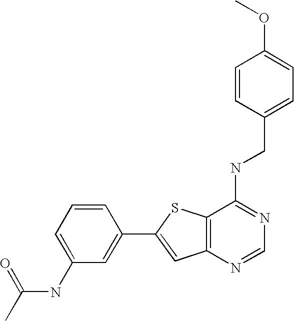 | 2.84 | 404 | 405 |
| 91 | 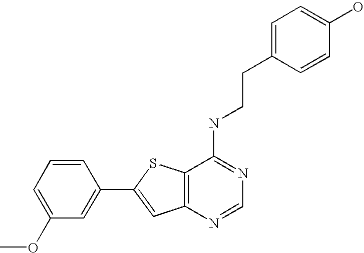 | 2.88 | 377 | 378 |
| 92 | 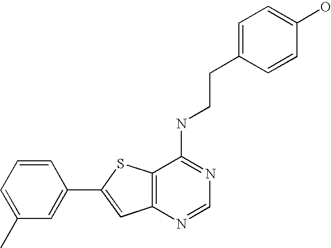 | 2.93 | 361 | 362 |
| 93 | 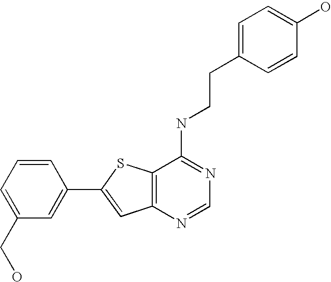 | 2.52 | 377 | 378 |
| 94 | 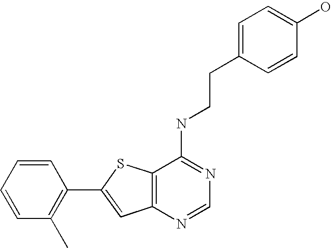 | 3.03 | 361 | 362 |
| 95 | 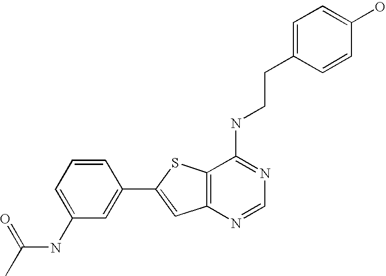 | 2.58 | 404 | 405 |
| 96 | 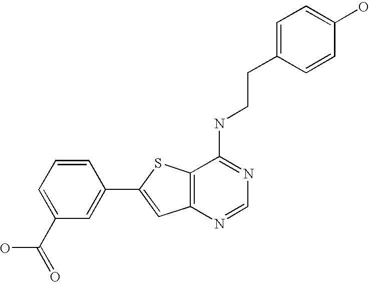 | 2.52 | 391 | 392 |
| 97 | 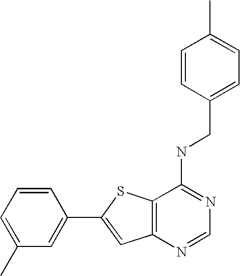 | 3.47 | 345 | 346 |
| 98 | 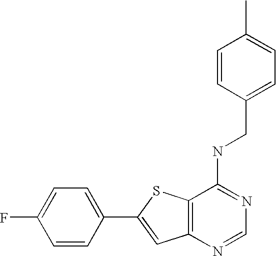 | 3.23 | 349 | 350 |
| 99 | 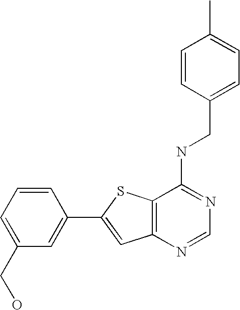 | 2.99 | 361 | 362 |
| 100 | 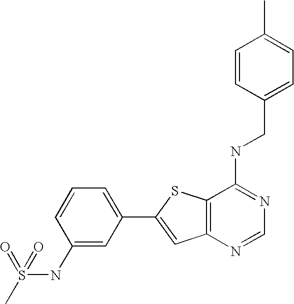 | 3.08 | 425 | 426 |
| 101 | 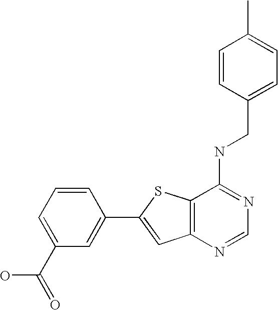 | 2.95 | 375 | 376 |

| Example | Structure | NMR (300 MHz, DMSO-d6) |
|---|---|---|
| 102 | 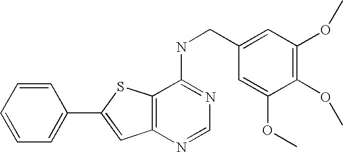 | δ = 3.59 (3H), 3.70 (6H),4.64 (2H), 6.68 (2H),7.38-7.54 (3H), 7.78-7.85(3H), 8.36 (1H), 8.43 (1H) |
| 103 | 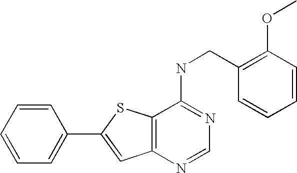 | δ = 3.81 (3H), 4.66 (2H),6.83 (1H), 6.97 (1H),7.13 (1H), 7.19 (1H),7.40-7.54 (3H), 7.78-7.86(3H), 8.28 (1H), 8.37 (1H) |
| 104 | 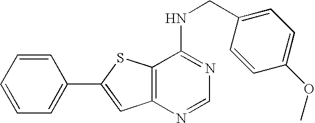 | δ = 3.68 (3H), 4.63 (2H),6.84 (2H), 7.26 (2H),7.40-7.52 (3H), 7.77-7.84(3H), 8.38 (1H), 8.40 (1H) |
| 105 | 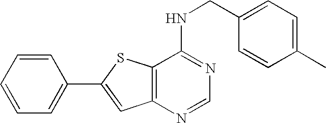 | δ = 2.22 (3H), 4.65 (2H),7.08 (2H), 7.21 (2H),7.39-7.52 (3H), 7.77-7.84(3H), 8.39 (1H), 8.40 (1H) |
| 106 | 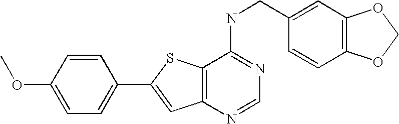 | δ = 3.79 (3H), 4.59 (2H),5.93 (2H), 6.80 (2H),6.89 (1H), 7.03 (2H), 7.65(1H), 7.74 (2H), 8.27 (1H),8.38 (1H) |

### BIOLOGICAL EXAMPLES

### 1. Detection of the Antagonism of the Human Prostaglandin E₂ (Subtype EP₂) Receptor Signal

### 1.1 Principle of Detection

The binding of PGE₂ to the EP₂ subtype of the human PGE₂ receptor induces activation of membrane-associated adenylate cyclases and leads to the formation of cAMP. In the presence of the phosphodiesterase inhibitor IBMX, cAMP which has accumulated due to this stimulation and been released by cell lysis is employed in a competitive detection method. In this assay, the cAMP in the lysate competes with cAMP-XL665 for binding of an Eu cryptate-labelled anti-cAMP antibody.

This results, in the absence of cellular cAMP, in a maximum signal which derives from coupling of this antibody to the cAMP-XL665 molecule. After excitation at 337 nm, this results in a FRET (fluorescence resonance energy transfer)-based, long-lived emission signal at 665 nm (and at 620 nM). The two signals are measured in a suitable measuring instrument with a time lag, i.e. after the background fluorescence has declined. Any increase in the low FRET signal caused by prostaglandin E₂ addition (measured as well ratio change=emission_{665 nm}/emission_{620 nm}*10 000) shows the effect of antagonists.

### 1.2. Detection Method

### 1.2.1 Antagonism Assay (Data for Each Well of a 384-Well Plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich #K-4002; including 750 μM 3-isobutyl-1-methylxanthine Sigma-Aldrich #I-7018), and then 15 μl thereof are transferred into a media-free cell culture plate which has been washed with KRSB shortly beforehand.

After preincubation at room temperature (RT) for 30 minutes, 5 μl of a 4×PGE₂ solution (11 nM) are added, and incubation is carried out in the presence of the agonist at RT for a further 60 min (volume: ˜20 μl) before the reaction is then stopped by adding 5 μl of lysis buffer and incubated at RT for a further 20 min (volume: ˜25 μl). The cell lysate is then transferred into a measuring plate and measured in accordance with the manufacturer's information (cyclic AMP kit Cisbio International #62AMPPEC).

### 1.2.2 Agonism Assay (Data for Each Well of a 384-Well Plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich #K-4002; including 750 μM 3-isobutyl-1-methylxanthine Sigma-Aldrich #I-7018), and then 15 μl thereof are transferred into a media-free cell culture plate which has been washed with KRSB shortly beforehand.

After incubation at room temperature (RT; volume: ˜15 μl) for 60 minutes, the reaction is then stopped by adding 5 μl of lysis buffer and incubated at RT for a further 20 min (volume: ˜20 μl). The cell lysate is then transferred into a measuring plate and measured in accordance with the manufacturer's instructions (cyclic AMP kit Cisbio International #62AMPPEC).

### 2. The EP₂ Subtype of the PGE₂ Receptor and the Preovulatory Cumulus Expansion

### 2.1. Background:

In the preovulatory antral follicle, the oocyte is surrounded by cumulus cells which form a dense ring of cells around the oocyte. After the LH peak (lutenizing hormone), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140(2):307-317). This cumulus expansion is an important component of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96(18):10501-6.) show a markedly reduced cumulus expansion and severe subfertility, demonstrating the importance of the prostanoid EP₂ receptor for this process.

### 2.2 Cumulus Expansion Assay In Vitro

Folliculogenesis is induced in immature female mice at an age of 14-18 days by a single dose (intraperitoneal) of 10 I.U. of PMSG (Pregnant Mare Serum Gonadotropin; Sigma G-4877, Lot 68H0909). 47-50 hours after the injection, the ovaries are removed and the cumulus-oocyte complexes are removed. The cumulus complex is not yet expanded at this stage.

The cumulus-oocyte complexes are then incubated with prostaglandin E₂ (PGE₂) (1 μM), vehicle control (ethanol) or test substances for 20-24 hours. Medium: alpha-MEM medium with 0.1 mM IBMX, pyruvates (0.23 mM) glutamines (2 mM), pen/strep 100 IU/ml pen. and 100 μg/ml strep. and HSA (8 mg/ml). Cumulus expansion is then established through the division into four stages (according to Vanderhyden et al. Dev Biol. 1990 August; 140(2):307-317).

TABLE 1
Example of the biological activity of the inventive compounds
(measured by means of the cAMP antagonism test):

| Substance according to | |
|---|---|
| example | Antagonism [IC₅₀ · μM] |
| 57 | 0.61 |
| 69 | 0.46 |
| 90 | 0.31 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding EP application No. 07 075 603.6, filed Jul. 12, 2007, and U.S. Provisional Application Ser. No. 60/949,341, filed Jul. 12, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula I 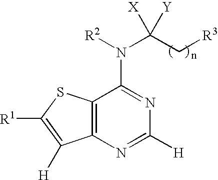
where
R¹ is a CH═CH-aryl group having a 6-10-membered, mono- or bicyclic aryl ring which is in each case unsubstituted or optionally mono- to trisubstituted,
a CH═CH-heteroaryl group having a 5-10-membered, mono- or bicyclic heteroaryl ring which is in each case unsubstituted or optionally mono- to trisubstituted,
a 6-10-membered, mono- or bicyclic aryl ring which is in each case unsubstituted or optionally mono- to trisubstituted,
a 5-10-membered, mono- or bicyclic heteroaryl ring which is in each case unsubstituted or optionally mono- to trisubstituted,
where the substituents may each be selected from the group of
halogen, cyano, CH₂CN, R⁴, OR⁴, CH₂OR⁴, OC(O)R⁴, S(O)ₙR⁴ where n=0, 1, 2,
SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
n is 0, 1 or 2,
X, Y are each independently hydrogen, a methyl group or together are a cyclopropyl ring,
R² is hydrogen, a C₁-C₄-alkyl group,
R³ is a 3-12-membered, mono-, bi- or tricyclic cycloalkyl radical,
a 6-10-membered, mono- or bicyclic aryl ring which is in each case unsubstituted or optionally mono- to trisubstituted,
a 5-10-membered, mono- or bicyclic heteroaryl ring which is in each case unsubstituted or optionally mono- to trisubstituted,
an 8-12-membered aryl-cycloalkyl or heteroaryl-cycloalkyl or aryl-heterocyclyl or heteroaryl-heterocyclyl group which is in each case unsubstituted or optionally mono- to trisubstituted,
where the substituents may be selected from the group of
halogen, cyano, CH₂CN, R⁴, OR⁴, CH₂OR⁴, OC(O)R⁴, S(O)ₙR⁴ where n=0, 1, 2,
SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
R⁴ is hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or -heteroaryl group, where the aryl radical is 6-membered and the heteroaryl radical is 5- or 6-membered,
R⁵ is hydrogen, a C₁-C₄-alkyl group,
R⁴, R⁵ together are a 3-6-membered ring,
and their stereoisomers, diastereomers, enantiomers, salts and their cyclodextrin clathrates.

2. Compounds according to claim 1, where
R¹ is a CH═CH-aryl group having a 6-10-membered, mono- or bicyclic aryl ring which is in each case unsubstituted or optionally mono- to trisubstituted,
a CH═CH-heteroaryl group having a 5-10-membered, mono- or bicyclic heteroaryl ring which is in each case unsubstituted or optionally mono- to trisubstituted,
a 6-10-membered, mono- or bicyclic aryl ring which is in each case unsubstituted or optionally mono- to trisubstituted,
a 5-10-membered, mono- or bicyclic heteroaryl ring which is in each case unsubstituted or optionally mono- to trisubstituted,
where the substituents may each be selected from the group of
halogen, cyano, CH₂CN, R⁴, OR⁴, CH₂OR⁴, OC(O)R⁴, S(O)NR⁴ where n=0, 1, 2,
SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
n is 0, 1 or 2,
X, Y are each independently hydrogen, a methyl group or together are a cyclopropyl ring,
R² is hydrogen, a methyl group,
R³ is a 3-12-membered, mono-, bi- or tricyclic cycloalkyl radical,
a 6-10-membered, mono- or bicyclic aryl ring which is in each case unsubstituted or optionally mono- to trisubstituted,
a 5-10-membered, mono- or bicyclic heteroaryl ring which is in each case unsubstituted or optionally mono- to trisubstituted,
an 8-12-membered aryl-cycloalkyl or heteroaryl-cycloalkyl or aryl-heterocyclyl or heteroaryl-heterocyclyl group which is in each case unsubstituted or optionally mono- to trisubstituted,
where the substituents may be selected from the group of
halogen, cyano, CH₂CN, R⁴, OR⁴, CH₂OR⁴, OC(O)R⁴, S(O)ₙR⁴ where n=0, 1, 2,
SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
R⁴ is hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or -heteroaryl group, where the aryl radical is 6-membered and the heteroaryl radical is 5- or 6-membered,
R⁵ is hydrogen, a C₁-C₄-alkyl group,
R⁴, R⁵ together form a 3-6-membered ring.

3. Compounds according to claim 1, where
R¹ is a CH═CH-aryl group having a 6-10-membered, mono- or bicyclic aryl ring which is in each case unsubstituted or optionally mono- to trisubstituted,
a CH═CH-heteroaryl group having a 5-10-membered, mono- or bicyclic heteroaryl ring which is in each case unsubstituted or optionally mono- to trisubstituted,
a 6-10-membered, mono- or bicyclic aryl ring which is in each case unsubstituted or optionally mono- to trisubstituted,
a 5-10-membered, mono- or bicyclic heteroaryl ring which is in each case unsubstituted or optionally mono- to trisubstituted,
where the substituents may each be selected from the group of
halogen, cyano, CH₂CN, R⁴, OR⁴, CH₂OR⁴, OC(O)R⁴, S(O)NR⁴ where n=0, 1, 2,
SO₂NHR⁴, SO₂NHC(O)R⁴, NR⁴R⁵, NHC(O)R⁴, NHSO₂R⁴, CH₂NR⁴R⁵, CH₂NHC(O)R⁴, C(OH)R⁴R⁵, C(O)R⁴, CO₂R⁴, C(O)NR⁴R⁵,
n is 0, 1 or 2,
X, Y are each independently hydrogen, a methyl group or together are a cyclopropyl ring,
R²: is hydrogen, a methyl group,
R³: is 3-6-membered, monocyclic cycloalkyl radical,
a 6-10-membered, mono- or bicyclic aryl ring which is in each case unsubstituted or optionally mono- to trisubstituted,
a 5-10-membered, mono- or bicyclic heteroaryl ring which is in each case unsubstituted or optionally mono- to trisubstituted,
an 8-12-membered aryl-cycloalkyl or heteroaryl-cycloalkyl or aryl-heterocyclyl or heteroaryl-heterocyclyl group which is in each case unsubstituted or optionally mono- to trisubstituted,
where the substituents may be selected from the group of
halogen, cyano, CH₂CN, R⁴, OR⁴, CH₂OR⁴, S(O)ₙR⁴ where n=0, 1, 2,
SO₂NHR⁴, SO₂NHC(O)R⁴, NHSO₂R⁴,
R⁴ is hydrogen, a C₁-C₄-alkyl group, a C₂-C₄-alkenyl group, a C₂-C₄-alkynyl group, a C₃-C₆-cycloalkyl group, a CH₂-C₃-C₆-cycloalkyl group, a 6-membered aryl ring, a 5-6-membered heteroaryl ring or a CH₂-aryl or -heteroaryl group, where the aryl radical is 6-membered and the heteroaryl radical is 5- or 6-membered,
R⁵ is hydrogen, a C₁-C₄-alkyl group,
R⁴, R⁵ together form a 3-6-membered ring.

4. Compounds according to claim 1, selected from a group which comprises the following compounds:
1. (6-Phenylthieno[3,2-d]pyrimidin-4-yl)thiophen-2-ylmethylamine
2. Cyclohexylmethyl-(6-phenylthieno[3,2-d]pyrimidin-4-yl)amine
3. (3-Morpholin-4-ylpropyl)-(6-phenylthieno[3,2-d]pyrimidin-4-yl)amine
4. Furan-2-ylmethyl-(6-phenylthieno[3,2-d]pyrimidin-4-yl)amine
5. (6-Phenylthieno[3,2-d]pyrimidin-4-yl)-(3-pyrrolidin-1-yl-propyl)amine
6. Phenethyl-(6-phenylthieno[3,2-d]pyrimidin-4-yl)amine
7. (6-Phenylthieno[3,2-d]pyrimidin-4-yl)pyridin-4-ylmethylamine
8. (2-Imidazol-1-yl-ethyl)-(6-phenylthieno[3,2-d]pyrimidin-4-yl)amine
9. 4-[2-(6-Phenylthieno[3,2-d]pyrimidin-4-ylamino)ethyl]phenol
10. Benzylmethyl-(6-phenylthieno[3,2-d]pyrimidin-4-yl)amine
11. Methyl-(6-phenylthieno[3,2-d]pyrimidin-4-yl)-(3,4,5-trimethoxybenzyl)amine
12. (6-Phenylthieno[3,2-d]pyrimidin-4-yl)-(tetrahydro-furan-2-ylmethyl)amine
13. (4-Fluorobenzyl)-(6-phenylthieno[3,2-d]pyrimidin-4-yl)amine
14. (2-Chloro-6-fluorobenzyl)-(6-phenylthieno[3,2-d]pyrimidin-4-yl)amine
15. (6-Phenylthieno[3,2-d]pyrimidin-4-yl)-(2-thiophen-2-yl-ethyl)amine
16. (6-Phenylthieno[3,2-d]pyrimidin-4-yl)-(2-pyrrolidin-1-yl-ethyl)amine
17. (6-Phenylthieno[3,2-d]pyrimidin-4-yl)-(2-pyridin-3-yl-ethyl)amine
18. (3-Phenylpropyl)-(6-phenylthieno[3,2-d]pyrimidin-4-yl)amine
19. [2-(4-Fluorophenyl)-ethyl]-(6-phenylthieno[3,2-d]pyrimidin-4-yl)amine
20. 4-[2-(6-Phenylthieno[3,2-d]pyrimidin-4-ylamino)ethyl]benzenesulphonamide
21. (2-Benzoimidazol-1-yl-ethyl)-(6-phenylthieno[3,2-d]pyrimidin-4-yl)amine
22. (3-Imidazol-1-yl-propyl)-(6-phenylthieno[3,2-d]pyrimidin-4-yl)amine
23. [2-(4-Fluorophenyl)-1,1-dimethylethyl]-(6-phenylthieno[3,2-d]pyrimidin-4-yl)amine
24. Benzo[1,3]dioxol-5-ylmethyl-(6-m-tolylthieno[3,2-d]pyrimidin-4-yl)amine
25. Benzo[1,3]dioxol-5-ylmethyl-(6-o-tolylthieno[3,2-d]pyrimidin-4-yl)amine
26. Benzo[1,3]dioxol-5-ylmethyl-[6-(4-fluorophenyl)thieno[3,2-d]pyrimidin-4-yl]amine
27. Benzo[1,3]dioxol-5-ylmethyl-[6-(3-fluorophenyl)thieno[3,2-d]pyrimidin-4-yl]amine
28. Benzo[1,3]dioxol-5-ylmethyl-[6-(4-chlorophenyl)thieno[3,2-d]pyrimidin-4-yl]amine
29. Benzo[1,3]dioxol-5-ylmethyl-[6-(2-fluorophenyl)thieno[3,2-d]pyrimidin-4-yl]amine
30. Benzo[1,3]dioxol-5-ylmethyl-(6-p-tolylthieno[3,2-d]pyrimidin-4-yl)amine
31. Benzo[1,3]dioxol-5-ylmethyl-[6-(3-methoxyphenyl)thieno[3,2-d]pyrimidin-4-yl]-amine
32. Benzo[1,3]dioxol-5-ylmethyl-(6-naphthalen-2-ylthieno[3,2-d]pyrimidin-4-yl)amine
33. Benzo[1,3]dioxol-5-ylmethyl-(6-biphenyl-4-ylthieno[3,2-d]pyrimidin-4-yl)amine
34. Benzo[1,3]dioxol-5-ylmethyl-(6-benzofuran-2-yl-thieno[3,2-d]pyrimidin-4-yl)amine
35. Benzo[1,3]dioxol-5-ylmethyl-[6-(3-chlorophenyl)thieno[3,2-d]pyrimidin-4-yl]amine
36. Benzo[1,3]dioxol-5-ylmethyl-[6-(4-dimethylaminophenyl)thieno[3,2-d]pyrimidin-4-yl]amine
37. (6-Benzo[b]thiophen-2-ylthieno[3,2-d]pyrimidin-4-yl)benzo[1,3]dioxol-5-ylmethylamine
38. Benzo[1,3]dioxol-5-ylmethyl-(6-naphthalen-1-ylthieno[3,2-d]pyrimidin-4-yl)amine
39. Benzo[1,3]dioxol-5-ylmethyl-[6-(3-fluoro-4-methylphenyl)thieno[3,2-d]pyrimidin-4-yl]amine
40. Benzo[1,3]dioxol-5-ylmethyl-[6-(2-fluoro-4-methylphenyl)thieno[3,2-d]pyrimidin-4-yl]amine
41. (4-{4-[(Benzo[1,3]dioxol-5-ylmethyl)amino]thieno[3,2-d]pyrimidin-6-yl}phenyl)methanol
42. 4-{4-[(Benzo[1,3]dioxol-5-ylmethyl)amino]thieno[3,2-d]pyrimidin-6-yl}benzoic acid
43. Benzo[1,3]dioxol-5-ylmethyl-[6-(3-trifluoromethylphenyl)thieno[3,2-d]pyrimidin-4-yl]amine
44. Benzo[1,3]dioxol-5-ylmethyl-[6-(5-fluoro-2-methoxyphenyl)thieno[3,2-d]pyrimidin-4-yl]amine
45. Benzo[1,3]dioxol-5-ylmethyl-[6-(4-tert-butylphenyl)thieno[3,2-d]pyrimidin-4-yl]amine
46. 1-(3-{4-[(Benzo[1,3]dioxol-5-ylmethyl)amino]thieno[3,2-d]pyrimidin-6-yl}-phenyl)ethanone
47. Benzo[1,3]dioxol-5-ylmethyl-[6-(3,4,5-trifluorophenyl)thieno[3,2-d]pyrimidin-4-yl]amine
48. Benzo[1,3]dioxol-5-ylmethyl-[6-(2,3,4-trimethoxyphenyl)thieno[3,2-d]pyrimidin-4-yl]amine
49. Benzo[1,3]dioxol-5-ylmethyl-[6-(3,4,5-trimethoxyphenyl)thieno[3,2-d]pyrimidin-4-yl]amine
50. Benzo[1,3]dioxol-5-ylmethyl-(6-biphenyl-3-ylthieno[3,2-d]pyrimidin-4-yl)amine
51. Benzo[1,3]dioxol-5-ylmethyl-[6-(3-isopropylphenyl)thieno[3,2-d]pyrimidin-4-yl]amine
52. (3-{4-[(Benzo[1,3]dioxol-5-ylmethyl)amino]thieno[3,2-d]pyrimidin-6-yl}phenyl)methanol
53. Benzo[1,3]dioxol-5-ylmethyl-[6-(3-methylsulphanylphenyl)thieno[3,2-d]pyrimidin-4-yl]amine
54. Benzo[1,3]dioxol-5-ylmethyl-[6-(2-chlorophenyl)thieno[3,2-d]pyrimidin-4-yl]amine
55. (6-Benzo[b]thiophen-3-yl-thieno[3,2-d]pyrimidin-4-yl)benzo[1,3]dioxol-5-ylmethylamine
56. Benzo[1,3]dioxol-5-ylmethyl-[6-((E)styryl)thieno[3,2-d]pyrimidin-4-yl]amine
57. Benzo[1,3]dioxol-5-ylmethyl-(6-quinolin-6-yl-thieno[3,2-d]pyrimidin-4-yl)amine
58. Benzo[1,3]dioxol-5-ylmethyl-[6-(3-pyrrolidin-1-ylphenyl)thieno[3,2-d]pyrimidin-4-yl]amine
59. 5-{4-[(Benzo[1,3]dioxol-5-ylmethyl)amino]thieno[3,2-d]pyrimidin-6-yl}-2-fluorobenzonitrile
60. Benzo[1,3]dioxol-5-ylmethyl-[6-(6-fluoro-5-methylpyridin-3-yl)thieno[3,2-d]pyrimidin-4-yl]amine
61. Benzo[1,3]dioxol-5-ylmethyl-[6-(6-methoxypyridin-3-yl)thieno[3,2-d]pyrimidin-4-yl]amine
62. Benzo[1,3]dioxol-5-ylmethyl-[6-(3-chloro-4-methylphenyl)thieno[3,2-d]pyrimidin-4-yl]amine
63. 4-{4-[(Benzo[1,3]dioxol-5-ylmethyl)amino]thieno[3,2-d]pyrimidin-6-yl}-N-methylbenzamide
64. (3-{4-[(Benzo[1,3]dioxol-5-ylmethyl)amino]thieno[3,2-d]pyrimidin-6-yl}phenyl)acetonitrile
65. 2-{4-[(Benzo[1,3]dioxol-5-ylmethyl)amino]thieno[3,2-d]pyrimidin-6-yl}benzamide
66. Benzo[1,3]dioxol-5-ylmethyl-[6-(3-fluoro-4-methoxyphenyl)thieno[3,2-d]pyrimidin-4-yl]amine
67. Benzo[1,3]dioxol-5-ylmethyl-[6-(3-chloro-4-fluorophenyl)thieno[3,2-d]pyrimidin-4-yl]amine
68. N-(3-{4-[(Benzo[1,3]dioxol-5-ylmethyl)amino]thieno[3,2-d]pyrimidin-6-yl}phenyl)methanesulphonamide
69. N-(3-{4-[(Benzo[1,3]dioxol-5-ylmethyl)amino]thieno[3,2-d]pyrimidin-6-yl}phenyl)acetamide
70. Benzo[1,3]dioxol-5-ylmethyl-[6-(5-chlorothiophen-2-yl)thieno[3,2-d]pyrimidin-4-yl]amine
71. Benzo[1,3]dioxol-5-ylmethyl-[6-(3,4-difluorophenyl)thieno[3,2-d]pyrimidin-4-yl]amine
72. Benzo[1,3]dioxol-5-ylmethyl-[6-(4-methoxy-3,5-dimethylphenyl)thieno[3,2-d]pyrimidin-4-yl]amine
73. Benzo[1,3]dioxol-5-ylmethyl-[6-(3,5-difluorophenyl)thieno[3,2-d]pyrimidin-4-yl]amine
74. Benzo[1,3]dioxol-5-ylmethyl-[6-(2,3-difluorophenyl)thieno[3,2-d]pyrimidin-4-yl]amine
75. Benzo[1,3]dioxol-5-ylmethyl-[6-(2,5-difluorophenyl)thieno[3,2-d]pyrimidin-4-yl]amine
76. Benzo[1,3]dioxol-5-ylmethyl-{6-[(E)-2-(4-fluorophenyl)vinyl]thieno[3,2-d]pyrimidin-4-yl}amine
77. Benzo[1,3]dioxol-5-ylmethyl-[6-(2-fluoro-3-methoxyphenyl)thieno[3,2-d]pyrimidin-4-yl]amine
78. (5-{4-[(Benzo[1,3]dioxol-5-ylmethyl)amino]thieno[3,2-d]pyrimidin-6-yl}thiophen-2-yl)methanol
79. Benzo[1,3]dioxol-5-ylmethyl-[6-(3-cyclopropylmethoxyphenyl)thieno[3,2-d]pyrimidin-4-yl]amine
80. Benzo[1,3]dioxol-5-ylmethyl-[6-(3-vinylphenyl)thieno[3,2-d]pyrimidin-4-yl]amine
81. Benzo[1,3]dioxol-5-ylmethyl-[6-(3,5-dimethylphenyl)thieno[3,2-d]pyrimidin-4-yl]amine
82. Benzo[1,3]dioxol-5-ylmethyl-(6-quinolin-3-ylthieno[3,2-d]pyrimidin-4-yl)amine
83. (4-Methoxybenzyl)-[6-(3-methoxyphenyl)thieno[3,2-d]pyrimidin-4-yl]amine
84. (4-Methoxybenzyl)-(6-m-tolylthieno[3,2-d]pyrimidin-4-yl)amine
85. [6-(4-Fluorophenyl)thieno[3,2-d]pyrimidin-4-yl]-(4-methoxybenzyl)amine
86. {3-[4-(4-Methoxybenzylamino)thieno[3,2-d]pyrimidin-6-yl]-phenyl}methanol
87. 4-[4-(4-Methoxybenzylamino)thieno[3,2-d]pyrimidin-6-yl]-N-methylbenzamide
88. (4-Methoxybenzyl)-(6-o-tolylthieno[3,2-d]pyrimidin-4-yl)amine
89. N-{3-[4-(4-Methoxybenzylamino)thieno[3,2-d]pyrimidin-6-yl]phenyl}-methanesulphonamide
90. N-{3-[4-(4-Methoxybenzylamino)thieno[3,2-d]pyrimidin-6-yl]phenyl}acetamide
91. 4-{2-[6-(3-Methoxyphenyl)thieno[3,2-d]pyrimidin-4-ylamino]ethyl}phenol
92. 4-[2-(6-m-Tolylthieno[3,2-d]pyrimidin-4-ylamino)ethyl]phenol
93. 4-{2-[6-(3-Hydroxymethylphenyl)thieno[3,2-d]pyrimidin-4-ylamino]ethyl}-phenol
94. 4-[2-(6-o-Tolylthieno[3,2-d]pyrimidin-4-ylamino)ethyl]phenol
95. N-(3-{4-[2-(4-Hydroxyphenyl)ethylamino]thieno[3,2-d]pyrimidin-6-yl}phenyl)acetamide
96. 3-{4-[2-(4-Hydroxyphenyl)ethylamino]thieno[3,2-d]pyrimidin-6-yl}benzoic acid
97. (4-Methylbenzyl)-(6-m-tolylthieno[3,2-d]pyrimidin-4-yl)amine
98. [6-(4-Fluorophenyl)thieno[3,2-d]pyrimidin-4-yl]-(4-methylbenzyl)amine
99. {3-[4-(4-Methylbenzylamino)thieno[3,2-d]pyrimidin-6-yl]phenyl}methanol
100. N-{3-[4-(4-Methylbenzylamino)thieno[3,2-d]pyrimidin-6-yl]phenyl}methanesulphonamide
101. 3-[4-(4-Methylbenzylamino)thieno[3,2-d]pyrimidin-6-yl]benzoic acid
102. (6-Phenylthieno[3,2-d]pyrimidin-4-yl)-(3,4,5-trimethoxybenzyl)amine
103. (2-Methoxybenzyl)-(6-phenylthieno[3,2-d]pyrimidin-4-yl)amine
104. (4-Methoxybenzyl)-(6-phenylthieno[3,2-d]pyrimidin-4-yl)amine
105. (4-Methylbenzyl)-(6-phenylthieno[3,2-d]pyrimidin-4-yl)amine
106. Benzo[1,3]dioxol-5-ylmethyl-[6-(4-methoxyphenyl)thieno[3,2-d]pyrimidin-4-yl]amine.

5. Medicaments which comprise at least one of the compounds of the formula I according to claim 1.

6. Medicaments according to claim 5 comprising suitable formulation and carrier substances.

7. Medicaments according to claim 5, characterized in that the medicament is used for the treatment and prophylaxis of disorders.

8. Use of the medicament according to claim 7 for treatment and prophylaxis of disorders which are connected to the EP₂ receptor.

9. Use of the medicament according to claim 7 for treatment and prophylaxis of fertility disorders.

10. Use of the medicament according to claim 7 for treatment and prophylaxis of menstrual complaints.

11. Use of the medicament according to claim 7 for treatment and prophylaxis of endometriosis.

12. Use of the medicament of the compounds according to claim 1 for modulation of the EP₂ receptor.

13. Use of the medicament according to claim 7 for treatment and prophylaxis of pain.

14. Use of the compounds according to claim 1 for fertility control/contraception.

15. Use of the medicament according to claim 7 for treatment and prophylaxis of osteoporosis.

16. Use of the medicament according to claim 7 for treatment and prophylaxis of cancer.

17. Use of the medicament according to claim 7 for treatment and prophylaxis of inflammation disorders, for example Crohn's disease.

18. Use of the compounds of the general formula I according to claim 1 in the form of a pharmaceutical preparation for enteral, parenteral, vaginal and oral administration.

19. Process for preparing the compounds according to claim 1, characterized in that the reaction of a thienopyrimidine Va or Vb with an amine III leads to the inventive compounds of the general formula I or the intermediates of the formula II, and, in a further step, the intermediates of the formula II are converted by the reaction with a boronic acid or a boronic ester of the general formula IV in a palladium(0)-catalysed reaction to further inventive compounds of the general formula I.

20. A method for treating or preventing a disorder connected to the EP₂ receptor comprising administering to a subject in need thereof an effective amount of a compound according to claim 1.
